(19) United States
(12) Patent Application Publication
(10) Pub. No.: US 2008/0187546 A1
(43) Pub. Date: Aug. 7, 2008

(54) Method for Replicating Influenza Virus in Culture

(76) Inventors: Terri L. Wasmoen, Omaha, NE (US); Peng Gao, Omaha, NE (US); Bradley Allen Eddy, Elkhorn, NE (US); Omar Yousif Abdelmagid, Kalamazoo, MI (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/956,658

(22) Filed: Dec. 14, 2007

Related U.S. Application Data
(60) Provisional application No. 60/875,287, filed on Dec. 15, 2006.
(60) Provisional application No. 60/882,412, filed on Dec. 28, 2006.

(51) Int. Cl. A61K 39/395; C12N 7/02; A61K 39/145

(52) U.S. Cl. 424/159.1; 435/239; 424/209.1

(57) ABSTRACT

The invention is related to a method for selecting an influenza virus for growth on tissue culture cells to produce a tissue-culture adapted viral isolate. The invention also includes vaccines produced from the isolate.

### CROSS-REFERENCES TO RELATED APPLICATIONS

The present application claims priority to U.S. Application No. 60/875,287, filed Dec. 15, 2006 and U.S. Application No. 60/882,412, filed Dec. 28, 2006, both of which are incorporated herein by reference.

### BACKGROUND OF THE INVENTION

Influenza epidemics and pandemics have been recognized for several centuries and have resulted in considerable loss of life. Influenza virus is a segmented RNA-containing virus belonging to the family Orthomyxoviridae. The epidemics and pandemics are caused by the appearance of viruses with new envelope components for which there is little immunity in the population. These new components are often the result of mutation and/or mixing of human and animal influenza viruses.

Whereas the capsid of the influenza virus is somewhat pleomorphic, the outer surface is consistent for all viruses and consists of a lipid envelope from which projects prominent glycoprotein spikes of two types; hemagglutinin (HA or H) and neuraminidase (NA or N). There are three types of influenza viruses; A, B, and C. Only influenza A viruses are further classified by subtype on the basis of the two main surface glycoproteins HA and NA. Influenza A subtypes are further classified by strains. Influenza B viruses infect mammals only and cause disease in humans, but generally not as severe as A types. Influenza C viruses also infect mammals only, but cause only a very mild respiratory disorder in children. They are genetically and morphologically distinct from A and B types.

Influenza A viruses infect a wide variety of animals, including mammals e.g., humans, horses, dogs, swine, ferrets ad avians, e.g. ducks, chickens and turkeys. There are 16 known HA serotypes and 9 known NA serotypes. Birds are particularly important reservoirs, generating pools of genetically/antigenically diverse viruses which get transferred back to the human population via close contact between humans and animals. Pigs are permissive to both human and bird influenza strains. Because of this unusual feature, pigs are considered as “Mixing Vessels” allowing for genetic exchange between avian and human viruses when the same cell is infected with both types of virus.

The genome of the influenza virus consists of single strand (−)sense RNA in 8 segments (7 in Influenza C). The structure of the genome is known in great detail because of the tremendous amount of generic investigation (conventional and molecular) which has been done. Each segment encodes one or two viral proteins. Epidemics and pandemics are believed to be due to genetic change in the HA and NA proteins of the influenza virus in two different ways: antigenic drift and antigenic shift. Antigenic drift occurs constantly, whereas antigenic shift happens only occasionally. Influenza type A viruses undergo both kinds of changes; influenza type B viruses change only by the more gradual process of antigenic drift.

Antigenic drift refers to small, gradual changes that occur through point mutations in the two genes that contain the genetic material to produce the main surface proteins, HA and NA. Antigenic shift refers to an abrupt, major change to produce a novel influenza A virus subtype in humans that was not currently circulating among people. Antigenic shift can occur either through direct animal (poultry)-to-human transmission or through mixing of human influenza A and animal influenza A virus genes to create a new human influenza A subtype virus through a process called genetic reassortment. Antigenic shift results in a new human influenza A subtype. Genetic reassortment occurs when two different influenza viruses infect the same cell and share or trade one or more RNA segments. If the segment that is transferred is the HA, for example, this can result in the appearance of a new viral strain that is antigenically new entering a population with little or no immunity. The result can lead to an epidemic and or a pandemic.

The entry of influenza viruses into cells is facilitated by binding of the HA spikes to mucoproteins containing terminal N-acetyl neuraminic acid (NANA=sialic acid) groups. After binding, the particle is engulfed by endocytosis via coated pits into endocytotic vesicle and finally endosomes. These are acidified by the cell and at about pH 5.0, the HA monomers are cleaved by trypsin-like enzymes in the endosome to activate them for internalization. Once internalized the viral replication occurs and results in the symptoms of influenza.

There is considerable concern about recent outbreaks of influenza. A severe type of respiratory disease has been identified in dogs, which is due to Canine Influenza Virus (CIV). This respiratory disease has proven to be highly contagious. Moreover, CIV can cause 100% infection with 80% morbidity, and up to 5-8% mortality in severe infections. Since its first detection in 2004 in greyhound racing dogs (Crawford et al., Science 310(5747):482-485 (2005)) CIV has rapidly spread across the United States with at least 25 states reporting CIV outbreak, and twenty-seven states reporting CIV seroprevalence.

The serotype of the CIV causing the recent outbreak is H3N8. This CIV serotype was originally discovered in horses, and is believed to have crossed the species barrier into canines. It is probable that the absence of an effective vaccine against canine influenza virus plays a major role in the rapid and widespread dissemination in dogs of this virus.

Influenza A (H5N1, avian influenza) virus—also called “H5N1 virus”—is an influenza A virus subtype that occurs mainly in birds, is highly contagious among birds, and can be deadly to them. H5N1 virus does not usually infect people, but infections with these viruses have occurred in humans. To date, over 200 confirmed human cases that resulted in over 150 deaths have been reported in 10 countries, mainly in Asia. Fortunately, as of yet, the virus does not readily spread from birds to humans or from one human to another. However, this could happen with the result that an epidemic or pandemic could occur. The best strategy for prevention of morbidity and mortality associated with an epidemic or pandemic is vaccination.

The influenza vaccines presently administered to humans have a high benefit-to-cost ratio in terms of preventing hospitalizations and deaths, however, the world's annual production capacity for seasonal vaccine is limited and does not realistically cover the global high-risk population. The present vaccines are made in eggs using virus obtained from the World Health Organization (WHO) or the Centers for Disease Control (CDC), who provide the virus seeds for vaccine manufacture every year. Changes in the HA of circulating viruses (antigenic drift) require periodic replacement of the vaccine strains during interpandemic periods. The WHO publishes semiannual recommendations for the strains to be included for the Northern and Southern Hemispheres. To allow sufficient time for manufacture, the WHO determines in February which vaccine strains should be included in the following winter's vaccine. In general, 1 dose for adults contains the equivalent of 45 μl HA (15 μg each for 3 viruses). This dose is approximately the amount of purified virus obtained from the allantoic fluid of one infected embryonated egg. If 100 million doses of killed influenza virus vaccine are prepared, the manufacturer has to procure 100 million embryonated eggs. This makes vaccine production dependent on the timely availability of good quality embryonated eggs and the seed strains provided by the WHO/CDC. Most of the prototype seed strains are not easily grown to high titer even in embryonated eggs. To overcome this problem, government agencies first create high-yielding laboratory strains through classical reassortment with high-yielding laboratory strain A/PR/8/34 (in a 6:2 reassortment obtaining 6 segments from the A/PR/8/34 strain). Unfortunately this process can be difficult to do and may effect the antigenicity of the resulting vaccine. Therefore there is a need to provide alternative methods of manufacturing vaccines that protect against clinical diseases caused by influenza, particularly highly pathogenic strains such as H5N1. Furthermore, there remains a need to provide methods of manufacturing large quantities of life-saving influenza vaccines in a time period quick enough to effectively prevent possible epidemics and/or pandemics. The present invention addresses these and other needs.

The citation of any reference herein should not be construed as an admission that such reference is available as “prior art” to the instant application.

### BRIEF SUMMARY OF THE INVENTION

The present invention relates to vaccines for the prevention of influenza A and B infections. The vaccines and the related methods of the invention provide a number of advantages over prior art vaccines and methods. For example, the vaccines of the invention are produced using tissue culture cells instead of embryonated eggs. The inventive production methods save critical time by bypassing the classical vaccine manufacture procedure. In addition, the vaccines of the invention are useful for those that are allergic to egg material. The present invention also provides new immunogenic compositions that may be used in the vaccines. These new immunogenic compositions can be used to immunize animals, including avians, against influenza virus. In particular embodiments of the invention, the recipient of the vaccine is a mammal. In one aspect, the present invention provides a vaccine that protects canines against the canine respiratory disease due to Canine influenza Virus (CIV). In another aspect, the present invention provides a vaccine that protects humans against influenza virus strains naturally produced through genetic reassortment.

The invention provides influenza virus isolates that have been specifically adapted to grow in tissue culture cells. In a particular embodiment, the adapted influenza virus isolates are selected for their ability to grow on a chosen tissue culture cell line using limit dilution cloning. In one particular embodiment, a subpopulation of influenza virus adapted for growth on a cultured cell line is selected by serially diluting into a multiplicity of aliquots, a quantity of influenza virus that comprises a multiplicity of influenza subpopulations. The multiplicity of aliquots are then contacted with and/or grown in the cultured cell line. A subpopulation of influenza virus within the multiplicity of influenza subpopulations is identified as one that grows on the cultured cells at a low multiplicity of infection (MOI) and selected as the subpopulation of influenza virus that is adapted for growth on the cultured cell line. In related embodiments, the present invention provides methods that include contacting the tissue-culture adapted isolate with tissue culture cells, growing for a time sufficient to produce cytopathic effects (CPE). This method can also include harvesting the influenza virus. In some such embodiments, the limit dilution cloning process involves serially diluting an influenza virus isolate and contacting each dilution with cultured cells, growing the cells for a time sufficient to produce cytopathic effects (CPE), harvesting virus from the highest dilution that causes CPE, and repeating the process with the harvested virus. In some embodiments, the method also includes admixing the influenza virus isolate with an effective amount of trypsin before contacting the cultured cells. In some embodiments, the mixture is incubated for a time sufficient to allow the trypsin to cleave viral proteins, without detaching the cells from the substrate. The trypsin used for cleaving viral proteins can be type IX trypsin. In some cases, the step of contacting the tissue-culture adapted isolate with the tissue culture cells is carried out at a multiplicity of infection (MOI) of less than about 0.01, including less than about 0.001 and/or less than about 0.0001. In other cases, the influenza virus isolate is first tested on the tissue culture cells to determine the optimal MOI. The tissue culture cells can be mammalian embryonic kidney cells such as human embryonic kidney cells. The influenza virus can be an influenza A, B, or C virus. In one particular embodiment, the influenza A virus is an H5N1 strain. The influenza virus isolate can be obtained form any number of sources including from a nasal swab, a lung tissue, and/or can be provided by a third party, e.g., the WHO. In some embodiments the influenza virus isolate is initially grown in embryonated eggs to obtain a large inoculum for adaptation to tissue culture. Some methods include purifying the harvested virus. In one such method, the step of purifying is carried out using size exclusion chromatography. The methods of the invention can also involve admixing a second isolate of influenza virus with the first isolate prior to, during or subsequent to purification, such that the second isolate is a different strain from the first isolate. In some methods dose titration studies are performed prior to admixing the two viral isolates to determine a mixture that allows equal immunogenicity of the viral proteins. In some methods the influenza is inactivated. In some embodiments of this type, the influenza virus is treated with an amount of binary ethyleneimine effective to inactivate it. In some methods, the harvesting is performed when the hemagglutinin protein content is maximal.

Further embodiments include a vaccine prepared by harvesting the viral isolate prepared by the method of selecting an influenza virus for growth on tissue culture cells by titrating an influenza virus isolate using limit dilution cloning such that an influenza virus isolate that is adapted to the tissue culture cells is selected. In some embodiments the virus is prepared by inoculation of embryonated specific pathogen free chicken eggs by chorioallantoic (also called the allantoic cavity) or amniotic membrane inoculation routes prior to limited dilution cloning. In one embodiment the influenza virus is replicated first by inoculation via the amniotic membrane of embryonated eggs to obtain an inoculum for adaptation to tissue culture cells.

In addition, the invention provides methods of selecting an influenza virus for growth on human embryonic kidney cells. In one such method an influenza virus isolate is titrated using limit dilution cloning such that an influenza virus isolate that is adapted to the HEK cells is selected. This method can include contacting the HEK-adapted isolate with HEK cells and growing, the cells for a time sufficient to produce cytopathic effects (CPE). In a particular embodiment of this type, the resulting influenza virus is harvested. The invention also provides a vaccine that includes the influenza virus isolate obtained by these methods. The method can also include admixing the influenza virus isolate with an effective amount of type IX trypsin before contacting the cultured cells for a time sufficient to allow the trypsin to cleave viral proteins, without detaching the cells from the substrate. In one embodiment, the step of contacting the HEK-adapted isolate with the HEK cells is carried out at an MOI of less than about 0.001.

In some embodiments, the invention further provides vaccines comprising human influenza virus formulated at less than 4 μg of human influenza HA per dose. In a related embodiment the invention provides vaccines comprising human influenza virus formulated at less than 3 μg of human influenza HA per dose. In another embodiment the invention provides vaccines comprising human influenza virus formulated at less than 2 μg, of human influenza HA per dose. In still another embodiment, the invention provides vaccines comprising human influenza virus formulated at 1.5-3.5 μg, of human influenza HA per dose. In particular vaccine embodiments the adjuvant is an ISCOM. In other vaccine embodiments at least 70% of the viruses comprise HA that have the same amino acid sequence. In still other vaccine embodiments at least 80% of the viruses comprise HA that have the same amino acid sequence. In yet other vaccine embodiments at least 90% of the viruses comprise HA that have the same amino acid sequence. In still other vaccine embodiments greater than 95% fo the viruses comprise HA that have the same amino acid sequence.

The present invention further provides combination vaccines for eliciting protective immunity against influenza virus, e.g., canine influenza virus (CIV) and other diseases, e.g. other canine infectious diseases. The present invention further provides for a method of immunizing a mammal, for example a dog, cat, or horse against influenza. Methods of making and using the vaccines to the infectious diseases, e.g., canine infectious diseases are also provided.

In a particular embodiment an immunogenic composition of the present invention comprises an immunogenic composition comprising an inactivated CIV H3N8 and an adjuvant. Typically, the adjuvant comprises an oil in water emulsion. In one such embodiment, the adjuvant further comprises aluminum hydroxide. In a particular embodiment of this type, the adjuvant is Emunade®. In another embodiment the immunogenic composition is a vaccine.

The vaccine composition may include from about 100 hemagglutination units (HAU) to about 1500 HAU per dose. This can vary widely depending on the size and other health considerations of the individual receiving treatment. The composition is typically between 250 and 750 HAU per dose. In one embodiment, the vaccine composition includes about 500 HAU per dose.

Optionally, the vaccines of the present invention can also include a pharmaceutically acceptable immune stimulant, e.g. cytokines, growth factors, chemokines, supernatants from cell cultures of lymphocytes, monocyes, or cells from lymphoid organs, cell preparations and/or extracts from plants, bacteria or parasites, or mitogens.

The vaccines of the present invention may be administered by a route such as: parenteral administration, intramuscular injection, subcutaneous injection, peritoneal injection, intradermal injection, oral administration, intranasal administration, scarification and combinations thereof. In a preferred embodiment of the invention, the vaccine is administered by intramuscular injection.

The invention also provides serum obtained from a vaccinated animal that contains antibodies that bind to CIV H3N8 and the purified antibodies themselves. In a particular embodiment of the invention, the purified antibody that binds to CIV H3N8 is a chimeric antibody.

The present invention further provides combination vaccines that include one or more strains of inactivated CIV, e.g., CIV H3N8, in combination with one or more other canine pathogens and/or immunogens, including, e.g., immunogens for eliciting immunity to canine distemper virus; canine adenovirus; canine adenovirus type 2; canine parvovirus; canine parainfluenza virus; canine coronavirus: canine influenza virus; and/or Leptospira serovars, e.g. Leptospira kirschneri serovar grippotyphosa, Leptospira interrogans serovar canicola, Leptospira interrogans icterohaemorrhagiae, and/or Leptospira interrogans serovar pomona. Additional canine pathogens that can be added to a combination vaccine of the present invention include Bordetella bronchiseptica; Leishmania organisms such as Leishmania major and Leishmania infantum; Borrelia species (spp.) spirochetes, including B. burgdorferi sensu stricto (ss), B. burgdorferi ss, B. garinii, and B. afzelii; a Mycoplasma species (e.g., Mycoplasma cynos); rabies virus; and Ehrlichia canis.

The present invention provides for methods of growing CIV H3N8 in cultured cells. In some embodiments, the cultured cells are non-canine mammalian kidney cells. In one embodiment, the cells are Madin-Darby bovine kidney (MDBK) cells. In another embodiment, the cells are Vero cells.

In some embodiments the invention further provides vaccines comprising CIV H3N8 formulated at least than 500 HAU per dose. In these embodiments, the adjuvant is usually aluminum hydroxide, and at least 70%, typically at least 90%, of the HA has the same amino acid sequence. In other vaccine embodiments at least 80% of the viruses comprise HA that have the same amino acid sequence. In still other vaccine embodiments greater than 95% of the viruses comprise HA that have the same amino acid sequence.

These and other aspects of the present invention will be better appreciated by reference to the following Figures and the Detailed Description.

### BRIEF DESCRIPTION OF THE DRAWINGS

FIG. 1 shows the average of clinical scores following CIV challenge in dogs. Non-vaccinated control and vaccinated dogs were challenged with CIV and monitored daily from day −2 through 10 days post-challenge for clinical signs such as ocular and nasal discharge, sneezing, coughing, depression and dyspnea. The clinical signs were scored as per the guidelines described in Example 1 and average clinical scores for each treatment group were plotted against days.

FIG. 2 demonstrates post-challenge nasal CIV shedding in dogs. Non-vaccinated control and vaccinated dogs were challenged with CIV. Nasal swabs were collected on the day before challenge (day −1) to on fin that the dogs were CIV free. Nasal virus shedding was monitored in challenged dogs by collecting nasal swabs daily for 10 days (day 1 through 10 post-challenge) and performing titration on MDCK monolayers. The average virus titers for each treatment group, expressed as Log₁₀ TCID₅₀/mL, were calculated and plotted against days post-challenge.

### DETAILED DESCRIPTION OF THE INVENTION

Traditional methods of producing influenza vaccines involve growth of an isolate strain in embryonated hens' eggs at least partly because the use of eggs is cheap and efficient and because there is no readily apparent alternative choice or growth of influenza in large amounts. In the case of human influenza this is particularly true because many cell lines that can be used to propagate influenza virus have not been approved by the FDA for human vaccine manufacture and only very low titers have been obtained in tissue culture.

When the vaccine is made in eggs, it is generally made as follows. Initially, the virus is recovered from a throat swab or similar source and isolated in eggs. The initial isolation in egg is difficult, but the virus adapts to its egg host and subsequent propagation in eggs takes place relatively easily. After growth in the egg, the virus is purified and formalin or beta-propiolactone inactivated. Growing evidence suggests that the egg is not optimal for virus propagation. For example, the conventional laying flocks used in egg-based manufacture are at high risk for contaminating the virus preparation with endogenous viruses routinely found in these settings. Also, the separate inoculation and harvest of millions of eggs as well as the complicated downstream processing results in vast opportunities for environmental contaminants to be introduced into the virus preparation and is likely the reason there was a recall of vaccine in 2004. As mentioned previously, it is difficult to completely remove egg material and this can result in sensitivity to the vaccine. In addition, the process completely lacks flexibility if demand suddenly increases because of the logistical problems due to the non-availability of large quantities of suitable eggs. There is also evidence that growth in eggs can reduce the antigenicity of the virus. Consistently, growing influenza A or B viruses in eggs results in a heterogenous viral product that has a spectrum of HA mutations. In direct contrast, the corresponding growth in mammalian host cells results in influenza viruses that are structurally identical to those originally isolated (Rocha, et al. J. Gen. Virol 1993; 74:2513-2518). Moreover, influenza viruses grown in mammalian cells elicit neutralizing and HA inhibition antibodies in human sera more readily and with a higher titer than do their egg-grown counterparts (Oxford, et al. Bull WHO 1987; 65:181-187).

Unfortunately, whereas all influenza viral strains seem to grow in eggs, heretofore, many do not grow well in tissue culture cells, and of those that do grow in tissue culture cells often do Slot grow in the quantities necessary to produce an effective, vaccine. The present inventors now disclose that when influenza virus is grown in tissue culture using limiting dilution cloning, isolates can be produced that are tissue culture-adapted. Surprisingly, the inventor's discovered that replication of a virus resulting from inoculation through the amniotic membrane of embryonated eggs resulted in a virus population that could replicate and produce high levels of HA when propagated on tissue culture cells (e.g.—Vero cells). The resulting viral isolate produces an HA titer that is almost equal to that obtained using embryonated eggs and HA titer is one important measure of vaccine potential. Therefore, one important aspect of the present invention is directed to methods of producing viral isolates of influenza virus that are tissue-culture adapted and suitable for use in the production of influenza vaccines, particularly mammalian vaccines. The methods involve the use of limit dilution cloning to isolate and identify tissue-culture adapted viruses. The resulting isolate can be treated to produce a vaccine. Thus, the present invention is also directed to methods of producing improved Influenza virus vaccines.

To this end, methods are provided for selecting tissue-culture adapted influenza virus by titrating the virus using limit dilution cloning and repeating the process 2 or more times. In some methods, the tissue culture cell used is a HEK cell. Trypsin or an equivalent protease can be used to increase the efficiency of viral entry into the cells. Further methods involve titrating the trypsin to identify the best concentration for the trypsin lot used and for the cells used. Identification of the best multiplicity of infection (MOI) for each influenza virus used and for the specific cells also contributes to successful tissue culture propagation.

The isolated tissue-culture adapted virus can be used to produce a vaccine according to standard methods. In some embodiments, the vaccines include the use of the adjuvant ISCOM. In some embodiments, the vaccines include the use of the adjuvant aluminium hydroxide. When more than one viral strain or isolate is included in the vaccine, methods can involve mixing the two in an immunologically equal amount. Methods and compositions are provided herein for the tissue-culture adapted viral isolates and for vaccines made therefrom.

### I. Methods of Selecting Tissue Culture Adapted Virus

The source of virus used in the methods of the invention is not critical to the invention. For examples the virus can be obtained by isolation from an infected animal or patient as a seed, virus stock from WHO, by purchase from an appropriate agency (e.g. ATCC), or from research laboratories. In particular, CIV is known to cause severe respiratory disease including pneumonia. Thus, dogs showing these symptoms are useful sources. Appropriate specimens for isolating virus include: nasal wash aspirate nasopharyngeal swab, throat swab, broncheoalveolar lavage, tracheal aspirate, pleural fluid tap, sputum, cloacal smears, and autopsy specimens. Specimens from living animals optimally should be collected early, and in some cases, within 4 days after illness onset. Specimens can be collected in an appropriate transport media to be stored until use or used immediately. If stored, the virus can be kept at a reduced temperature, such as at 4° C. to ensure viability. To isolate the influenza virus from the specimen, large contaminants can be removed (for example, by centrifugation) and the supernatant inoculated onto a variety of cells at a variety of dilutions. Alternatively the virus from the animal can initially be grown in hens eggs. Methods can be used to confirm that the virus isolate is indeed influenza at any point in the process.

To confirm the presence of the desired influenza virus at any time in the process of preparing a tissue-culture adapted isolate, a variety of screening methods can be used. The virus can be screened by assaying using any known and/or suitable assay for influenza virus. Such assays include (alone or in combination), viral replication, quantitative and/or qualitative measurement of inactivation (e.g., by antisera), transcription, replication, translation, virion incorporation, virulence, HA or NA activity, viral yield, and/or morphogenesis, using such methods as reverse genetics, reassortment, complementation, and/or infection.

A. Tissue Culture Cells

Any mammalian host cells that allow the growth of influenza virus can be used for the methods herein. Typically, the host cells suitably exclude adventitious agents and are of a passage number that can be certified according to the WHO requirement for vaccine production. A number of cell lines can be used to isolate and propagate influenza viruses. Some cell lines that have been used include: Vero cells (monkey kidney cells), MDCK cells (Madin-Darby canine kidney cells), BHK-21 cells (baby hamster kidney) and BSC (monkey kidney cell) and HEK cells hum an embryonic kidney cells). Thus, any tissue culture cell that allows the growth of influenza virus can be used. Suitable cells include, but are not limited to: Vero, MDBK, BK21, CV-1, and any mammalian embryonic kidney cell (e.g. HEK). In some embodiments, Vero cells or mammalian embryonic kidney cells are used. In some embodiments, human embryonic kidney cells (HEK cells) are used.

The appropriate tissue culture medium for propagation of the aforementioned cell lines will be known to those of skill in the art. Such medium may contain an appropriate serum (e.g., fetal bovine serum) at a concentration up to 20% v/v. It will be appreciated by those of skill, that a medium containing less than 20% v/v serum (2-5% v/v) can be used to propagate the aforementioned cell lines.

B. Optimizing the Trypsin for the Cells

In order to grow a high titer stock of the virus in cell cultures, an appropriate amount of protease can be used to activate the hemagglutinin for internalization into cells. The protease containing solution can be added to the isolate directly or the isolate can be diluted into the protease for growth of an isolate for production of a vaccine. The protease can be used to dilute the virus to the appropriate MOI for growth.

The protease can be any protease that is capable of activating the HA for internalization without damaging viral proteins such that they cannot grow and/or infect cells. Such proteases include, but are not limited to, prokaryotic protease, pronase, trypsin, and subtilisin (A), for example, Trysin IX.

The amount of protease to be used should be enough to activate the virus with very little toxic effect to the cells. Toxic effect can be analyzed by identifying characteristics of damage to the cell such as detachment from the plate or substrate, presence of cellular debris, appearance of dead cells and lack of viable cells. Thus, an “effective amount of trypsin” is one which, when used for a time sufficient, allows the trypsin to cleave viral proteins, without detaching the cells from the substrate or causing other toxic effects. Titrating the protease can also be used to increase production of active virus in tissue culture. Titration involves identifying the maximum amount of trypsin that is minimally damaging to the cells. This amount can vary with the tissue culture cells used and with the protease lot. Therefore, new protease lots can be titrated to establish the optimal level prior to using and each protease can be titrated for each tissue culture cell. Titration involves inoculating the tissue culture cells with step-wise dilutions of the protease and incubating them or an appropriate time. For example, half-step dilutions (10^{-0.5}) of protease may be used. Incubation time will vary with the cell line, but, typically will be between about 2 days and 7 days. The protease level can be determined using the typical incubation time for the cells being used. For example, if a 4 day incubation is best for the influenza virus, the protease can be tested by incubating for about 4 days in the presence of protease alone. The lowest dilution of protease that has no toxicity or very little toxicity for the cells can then be used. The range of trypsin concentrations that can be used in mammalian tissue culture cells, for example Vero cells, is from about 0.5 μg/mL to about 10 μg/mL, but more commonly about 2.5 μg/mL of medium.

Once the optimal level of protease is identified, the virus can be diluted in the infection medium with an appropriate amount of protease, (e.g., trypsin) such that the optimal level is reached when the virus is added to the cellular media. The optimal amount of trypsin can be used for the limit dilution cloning to produce a tissue-culture adapted isolate as well as growth and harvesting of the isolate.

C. Limiting Dilution Cloning

A typical virus culture is heterogeneous. Thus, for example the individual viral particles in the well of a microtiter plate can vary with respect to infectivity, replication, and the like. Serial dilution is used to select subpopulations of viruses in a culture, subpopulations that are best adapted to cells, for example. Serial dilution involves diluting a virus culture serially up to extinction, for example, to determine the best MOI. Typically this involves a series of 10-fold dilutions, but can vary depending upon the titer of virus.

Typically limit dilution is used to identify the highest dilution which produces viral effect on the cell. The viral effect can be a cytopathic effect (CPE). Cytopathic effects are any effects on the cell caused by influenza virus infection. These include, but are not limited to: cell rounding, degeneration, sloughing, apoptosis, induction of reactive oxygen species (ROS), cells becoming granular and then fragmented, and detachment of cells from a support (such as a tissue culture dish). The well of the highest dilution is harvested. This harvested virus is then diluted to extinction and the process is repeated. Typically, serial 10-fold dilutions are made in an appropriate medium (with or without trypsin), and 0.2 mL of each dilution is added to a plate or wells of a microtiter plate containing the tissue culture cells and incubated for a time sufficient to identify CPE of the cells. Because serum inactivates trypsin the medium typically does not contain serum. The well or plate containing the highest dilution that causes CPE is harvested, then diluted and the process is repeated. The process is typically repeated at least two times, but can be repeated up to 5 times. In some cases, the process is repeated 3 times.

Virus cultures produced by the methods of the invention are characterized by homogeneity of the sequence of the HA proteins in the viruses. A number of methods can be used to measure the degree of sequence homogeneity. For example, sequence the HA proteins themselves or by sequencing the RNA encoding such proteins. Typically, the viruses preparations produced by the methods of the invention will contain viruses in which at least 70% of the HA proteins have the same amino acid sequence. In some embodiments at 80%, or at least 90% of the HA proteins have the same amino acid sequence.

D. Methods of Testing Influenza Virus

Tests can be performed to confirm activity and the presence of influenza virus at any time in the process for the methods herein. For example, hemagglutination can be identified as follows. If the virus has a surface HA protein it can attach to RBCs and agglutinate them. If the concentration of virus in a sample is high, when the sample is mixed with RBCs, a lattice of viruses and RBCs will be formed. This phenomenon is called hemagglutination. It is a simple way to detect the presence and titer of viruses that hemagglutinate such as influenza viruses. If there is not enough virus in the sample to hemagglutinate the RBCs, they form a pellet at the bottom of the well. The highest dilution showing complete hemagglutination is taken as the end point. The viral titer is expressed in HA units (HAU), which are the inverse of the dilution per milliliter. For example if there is complete hemagglutination in the well, at a 1/32 dilution in 50 μLs, but not in the well with the next highest dilution, the titer of the virus is 32 HAU per 50 μLs or 640 HAU per mL.

Other assays that can be used to identify and quantify influenza virus include identification of CPE (as discussed herein), Western blot, ELSA, PCR, and other methods for identification of influenza virus using antibodies and/or probes that are specific for some part of the virus, particularly the HA antigen.

### II. Methods of Growing and Harvesting

After production of a virus isolate, the isolate can be harvested. Standard methods can be used. The harvested isolate can be stored for future use or used to prepuce a vaccine using standard methods. The virus can be harvested when a maximum amount of virus is produced; when a maximal amount of hemagglutinin is produced, as measured by HA assay; and, or when the cells are lysed.

After obtaining a tissue-culture adapted isolate, the isolate can be grown and harvested. The method of growing and harvesting the tissue-culture adapted virus is not critical to the invention and standard methods can be used. However, in some embodiments, the virus is grown in the tissue culture cell it is best adapted to. The harvested viral isolate can be stored for future use or used to produce a vaccine using standard methods. The virus can be grown on the appropriate tissue culture cells by adding the virus at an appropriate MOI in the appropriate amount of protease (e.g., trypsin) to cells for a time sufficient to produce a high titer of virus and/or until the cells are lysed. The virus can be harvested when a maximum amount of virus is produced, when a maximal amount of hemagglutin is produced and, or when the cells are lysed. It has been found herein that an optimal pre-growth of the virus in eggs (in the allantoic cavity or inoculation through the amniotic membrane) can increase the adaptation of virus in the tissue culture cells.

A. Pre-Growth in Eggs

Passage in egg cultures has been shown to facilitate the adaptation of virus in the tissue culture cell. Thus, it may be desirable to pre-grow the viral isolate in embryonated hens' eggs according to standard technologies. For example, the virus is injected into the allantoic cavity or via the amniotic membrane of 9-12 day old embryonated eggs and allowed to multiply for about three days. Then the allantoic or amniotic fluid is collected and the collected material can be grown in tissue culture at the appropriate MOI for use in production of a vaccine. Alternatively, the collected material can be directly used in limit dilution cloning. It has been found that inoculation of the eggs through the amniotic membrane enriches for virus that can replicate and can produce high levels of HA protein when grown in Vero cells.

B. MOI

It was identified herein that a low MOI results in a better and/or higher titer viral isolate. Without limitation to a specific theory, it is believed Teat a low MOI reduces the amount of defective virus particles and results in a more efficient infection process. In some embodiments, the MOI used is less than about 0.01 (one virus per 100 cells). In other embodiments, the MOI is less than about 0.003. In some embodiments, the MOI is less than about 0.001. The MOI can be chosen as being the lowest MOI that results in a high titer of virus and/or that lyses the cells in about 3 to 4 days.

### III. Vaccine Production

Once a desired isolate is obtained from the tissue-culture adapted virus, the virus can be used to produce a vaccine. Many types of viral vaccines are known, including but not limited to attenuated, inactivated, subunit, and split vaccines.

A. Methods of Producing Attenuated Virus

Attenuated vaccines are live viral vaccines that have been attenuated or changed to no longer cause disease. These can be produced in many ways, for example, growth in tissue culture for repeated generations and genetic manipulation to mutate or remove genes involved in pathogenicity. The tissue-culture adapted isolate can be used to produce an attenuated virus using standard methods. For example, once viral genes and/or proteins are identified that are involved in pathogenicity or involved in the disease manifestation, these can be mutated or changed such that the virus is still able to infect and replicate within a cell, but it cannot cause disease. An example of this is to mutagenize the HA1/HA2 cleavage site. The tissue-culture adapted virus can be attenuated using any standard methods, for example cold adapting the virus.

After production of the attenuated virus, the vaccine can be prepared using standard methods (for example, the methods herein). The virus can be purified using standard methods, for example using size exclusion chromatography. A vaccine can then be prepared using standard adjuvants and vaccine preparations, for example, ISCOM, nano-beads, mineral oil, vegetable oil, aluminum hydroxide, saponin, non-ionic detergents, squalene, and block copolymers can be used alone or in combination as adjutants. Current commercial vaccines in the United States and Europe do not contain any adjuvant (both live and killed vaccines) and this is partly why such a high concentration of HA (15 μg of HA per virus strain, 45 μg of HA for trivalent formulation) is needed in the vaccine.

B. Methods of Producing Inactivated, Subunit, and Split Virus Vaccines

Once a desired virus is obtained, it can be used to produce an immunogenic composition, for example, a vaccine. Examples of “killed” vaccines are inactivated, split and subunit vaccines. These can be prepared to treat influenza using standard methods.

For example, subunit vaccines generally involve isolating only the part of the virus that activates the immune system. In the case of Influenza, subunit vaccines have been prepared using purified HA and NA, but any mixture of viral proteins can be used to produce a subunit vaccine. Generally, the viral protein, such as HA is extracted from recombinant virus forms and the subunit vaccine is formulated to contain a mixture of these viral proteins from strains recommended by WHO. For example, the 1995-1996 vaccine contained the HA and NA from two A strains and one B strain (A/Singapore/6/81 (H1N1); A/Johannesburg/33/94 (H3N2); and B/Beijing/84/93). For H3N8 CIV, the H3 and/or N8 antigens can be used.

Generally, the viral protein(s) are extracted from the virus and the subunit vaccine is formulated to contain a mixture of these viral proteins. Proteins can be isolated from a tissue culture adapted viral isolate for a subunit vaccine using standard methods. Alternatively, the proteins may be produced using recombinant techniques. Techniques for producing a particular protein are known in the art.

Split vaccines generally involve treating enveloped viruses with detergent to solubilize the proteins therein. In the case of influenza virus, HA and NA become solubilized. For example, nonionic detergents such as Triton X-100 can be used for producing split vaccines.

Inactivated viral vaccines are prepared by inactivating the harvested virus and formulating it using known methods for use as a vaccine to induce an immune response in a mammal. The inactivation step, purification of subunits, and/or splitting can be performed before or after purification of the virus by size exclusion. For example, production of an inactivated vaccine, may involve removal of cellular material, inactivation of virus, purification and solubilization of the viral envelope. Other embodiments may involve purification of virus and then inactivation for example using formaldehyde.

Once prepared, any of the vaccines (for example, attenuated, split, subunit or inactivated) can be tested to identify that the virus and/or vaccine has maintained similar antigenicity, produces a serological response in a mammal, and/or provides protection from disease in a mammal.

C. Further Processing of Harvested Virus

1. Clarification of Harvested Virus

After harvesting and/or after inactivation of the harvested virus, the cellular material and other interfering materials can be removed, for example, by sedimenting to remove the microcarriers and concentrating the supernatant by ultrafiltration. Influenza grown in tissue culture cells will contain host cellular proteins. Some further clarification of the supernatant may be needed. Cellular DNA can be removed by enzymatic treatment (e.g.). After the initial removal of interfering material, virus can be inactivated using standard methods. Alternatively, inactivation can be performed after further purification by, for example, size exclusion chromatography.

2. Inactivation of Virus

The influenza virus may be inactivated in any number of ways and by any number of agents. The method of inactivation is not critical to the invention. Inactivation can occur after contaminating or interfering material is removed. Inactivation can include the use of known inactivating agents. Such inactivating agents include, but are not limited to; UV irradiation, formaldehyde, glutaraldehyde, binary ethyleneimine (BEI), and beta-propiolactone. In some embodiments BEI is used because it is known to destroy the viral nucleic acid without damaging the viral proteins. In addition, BEI is not affected by protein content and temperature. Inactivating agents are used at a concentration high enough to inactivate every viral particle in the solution. For example, BEI can be used at a final concentration of between about 0.5 and 10 mM, including but not limited to; 1.5, 3, 4, 5, and 6 mM and including ranges of about 1 to 6, and 1 to 3 mM. In one embodiment, the BEI is used at a concentration of about 6 mM. Typically, the BEI is used at a concentration of about 1.5 mM and incubated at 37° C. for 48 hours. In the preparation of vaccines to CIV, BEI can be used at a final concentration of between about 0.5 and at 10 mM usually between 4 to 8 and often between 5 to 7 mM. In one embodiment, the BEI is used at a concentration of about 6 mM. In some embodiments inactivation occurs at the appropriate pH and temperature for the inactivating agent. The pH and temperature can be chosen to ensure the resulting inactivated virus is still immunogenic. Inactivation can proceed with an appropriate amount of agitation to ensure that the agent contacts all virus particles in the solution.

After inactivation, the inactivating agents can be removed using methods including, but not limited to inactivation of the inactivating agent, precipitation of the inactivating agent, filtration of the inactivating agent, and chromatography, or a mixture of these methods. For example, BEI can be inactivated by the addition of sodium thiosulfate. Residual BEI can also be separated from virus/viral proteins using size exclusion methods. Once innocuity (absence of live virus) is confirmed, the viral solution can be further processed to produce a vaccine.

3. Further Processing

The viral solution can be further processed, for example, to remove contaminants, further concentrate the virus and provide for a stronger immune response. Some examples of further processing include initial removal of cellular material, removal of cellular DNA, concentration, and formulation in adjuvant using standard methods. Influenza grown in tissue culture cells will contain host cellular proteins. For example, influenza propagated on human embryonic kidney (HEK) cells will possess HEK proteins or bovine or simian proteins if grown in MDBK or VERO cells respectively. These proteins can be detected by methods known to those of skill in the art. Many methods are known for removal of DNA, including addition of a variety of DNase enzymes known to degrade cellular DNA, for example, Benzonase. An initial concentration step can be performed to provide the viral solution at a concentration best suited for additional purification by chromatography. This can be done using any standard methods, including but not limited to ultra filtration using a membrane having a molecular weight cut off of about 100K (e.g. polysulfone membrane with a MWCO of 100 K). The viral solution can be concentrated up to about 100 fold, including but not limited to 90 fold, 80 fold, 70 fold, 60 fold, 50 fold, 40 fold, 30 fold, 20 fold, 10 fold, and 5 fold. In some embodiments, the viral solution is concentrated up to about 50 fold, but more typically including 20 fold and 30 fold.

4. Purification

The virus can be purified using standard methods such as density centrifugation. In some embodiments, the virus is purified by size exclusion gel chromatography. One advantage of using size exclusion is that the yields are better than when using density centrifugation. Any size exclusion gel can be used that results in purification of virus. Any standard gel can be used, such as Sepharose gel (e.g. Sepharose CL-2B). In some embodiments, the column is about 70 to 120 cm in length to achieve the required separation, including but not limited to about 80, 90, 100, and 110. In other embodiments, the column is from about 80 to 100 cm in length, for example the column is about 90 cm in length. In some embodiments, the length is achieved by multiple columns in series, for example two 45 cm columns or three 30 cm columns (e.g., a column 30-32 cm in length by 30 cm in diameter). The concentrated virus can be applied using standard methods, for example, the virus is applied at 5-10% of the column volume (CV), typically 5-7% CV. The viral peak from the column can then be collected and further concentrated using standard methods, for example ultra filtration. In some embodiments, 2 to 3 columns in series having a total of 90 cm in length are used and the final peak is pooled and concentrated by ultrafiltration.

5. Solubilization of the Envelop Proteins

The concentration virus peak material can be solubilized using standard methods, such as, with a non-ionic detergent. The solubilization can be performed to prepare the material for formulation of ISCOMS (see below). Examples of non-ionic detergents, includes but are not limited to, Nonanoyl-N-Methylfucamide (Mega 9), Triton X-100, Octylglucoside, Digitonin, C12E8, Lubrol, Nonidet P-40, and Tween (for example Tween 20, 80 or 120). After solubilization, the virus can be used to produce a vaccine, and/or an adjuvant can be added. For example, for production of an ISCOM adjuvant, a lipid mixture can be added to assist ISCOM formation. The lipid mixture can include a phosphatidyl choline and synthetic cholesterol. In some embodiments, the virus is disrupted with Mega 9 at room temperature with stirring and then the lipid mixture (phosphatidyl choline and cholesterol) can be added and stirring continued.

6. Formation of Adjuvants

Appropriate adjuvants can be added to the vaccine and/or pharmaceutical composition. Examples of adjuvants include those containing an oil and water emulsion, as well as those that further comprise aluminum hydroxide. In the latter case, aluminum hydroxide from a commercial source can be used, for example, Alhydrogel, (Superfos Biosector, Frederikssund, Denmark) and Rehydrogel (Reheis Inc.). The oil and water emulsion typically comprises mineral oil or metabolizable oils (e.g., vegetable oil, fish oil). N on-ionic detergents or surfactants may be used as emulsifiers. Examples of emulsifiers include Tween 80/Span 80, Arlecel 80/Tween 80, and Montanides (Seppic, Paris, France). In the case of adjuvant emulsions, generally 5-25% of the volume is oil and 75-95% of the volume is aqueous. In some embodiments, the adjuvant emulsion is 20% oil and 80% aqueous by volume. The amount of aluminum hydroxide is usually between about 5% and 15% of the aqueous phase. In some embodiments, Emunade® is the adjuvant.

For some embodiments, ISCOM is used as an adjuvant. ISCOM is an acronym for Immune Stimulating Complex and the technology was described by Morein et al. (Nature 308:457-460 (1984)). ISCOM's are a novel vaccine delivery system and are unlike the conventional adjuvant techniques. An ISCOM can conveniently formed in one of two ways. In some embodiments, the antigen is physically incorporated in the structure during its formulation. In other embodiments, an ISCOM-matrix (as supplied by, for example, Isconova) does not contain antigen but is mixed with the antigen of choice by the end-user prior to immunization. After mixing, the antigens are present in solution with the ISCOM-matrix but are not physically incorporated into the structure.

Generally, in an ISCOM, purified antigens are presented in a multimeric form based on the ability of Quil A to spontaneously form micelles at a critical concentration and by a hydrophobic/hydrophile link, entrap the purified immunogens. These micellar structures are in the order of 35 nm in size and are easily recognized by the immune system. Unlike conventional depot adjuvants, ISCOMS are rapidly cleared from the injection site and illicit local, humoral and cell-mediated immune responses. In particular embodiments, ISCOMs are formed as follows. The virus is solubilized using standard methods such as with a none Lubrol Tween-80). A lipid mixture is added to assist ISCOM formation. The lipid mixture can include a phosphatidyl choline and a synthetic cholesterol. In some embodiments, the mixture is first treated with non-ionic detergent at room temperature with stirring, then the lipid mixture (equal parts phosphatidyl choline and cholesterol, for example) is added and stirring continued. Quil A (a purified glycoside of saponin) is added to the virus lipid mixture and stirring is continued. The Quil A can be added to give a final concentration of Quil A of about 0.01 to 0.1% f including but not limited to 0.02, 0.03, 0.04, 0.05, 0.06, 0.07, 0.08, and 0.09. In some embodiments, the final concentration is about 0.05%. The non-ionic detergent is removed (for example, by diafiltration with ammonium acetate). The matrix of the ISCOM is formed by Quit A. The morphology of an ISCOM particle, as viewed by electron microscopy, shows a typical cage like structure of approximately 35 nm in size. The ISCOM formation stage can be refined by the use of tangential flow diafiltration. ISCOMs present purified antigens in a multimeric form based on the ability of Quil A to spontaneously form micelles at a critical concentration and by a hydrophobic/hydrophilic link that entrap the purified antigens. Formation of ISCOMs can be verified by electron microscopy to verify that the typical cage-like structures have been formed. The immune response from an ISCOM presentation was shown to be at least ten times better than from a similar antigen payload presented as micelles of aggregated membrane protein alone. ISCOMs were also found to elicit a cell mediated response, not seen with conventional whole virus vaccines. In some embodiments, the final concentration is about 0.05%.

Immune stimulants may also be added to the vaccine and/or pharmaceutical composition. Immune stimulants include: cytokines, growth factors, chemokines, supernatants from cell cultures of lymphocytes, monocytes, or cells from lymphoid organs, cell preparations and/or extracts from plants, cell preparation and, or extracts from bacteria, parasites, or mitogens, and novel nucleic acids derived from other viruses ant, or other sources (e.g. double stranded RNA, CpG) block co-polymers, nano-beads, or other compounds known in the art, used alone or in combination.

Particular examples of adjuvants and other immune stimulants include, but are not limited to, lysolecithin; glycosides (e.g., saponin and saponin derivatives such as Quil A or GPI-0100); cationic surfactants (e.g. DDA); quaternary hydrocarbon ammonium halogenides; pluronic polyols; polyanions and polyatomic ions; polyacrylic acids, non-ionic block polymers (e.g., Pluronic F-127); and MDP (e.g. N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine).

D. Efficacy of Vaccines

Methods are well-known in the art for determining whether a subunit, attenuated, split, and/or inactivated virus vaccine has maintained similar antigenicity to that of the clinical isolate or tissue culture adapted isolate derived there from. Such known methods include the use of antisera or antibodies, HA and NA activity and inhibition and DNA screening (such as probe hybridization or PCR) to confirm that donor genes encoding the antigenic determinants are present in the inactivated virus. Methods of identifying whether the vaccine induces a serological response are also well known in the art and include immunization of a test animal with the vaccine, followed by inoculation with a disease-causing virus and identification of the presence or absence of symptoms of the disease. Thus, efficacy of the influenza vaccine can be tested in animals, usually ferrets, mice, and guinea pigs are used. Antibody titer can be tested using Hemagglutinatin inhibition (HI) or Neuraminidase inhibition (NI) method, or testing for virus neutralizing antibodies (micro neutralization test) in tissue culture, and such methods that are generally known. Challenge studies can provide important information to evaluate the vaccine.

Pharmaceutical compositions and/or vaccines suitable for treatment include virus or viral subunit in admixture with sterile aqueous or non-aqueous solutions. The process of producing a pharmaceutical composition and, or vaccine can involve isolating a tissue culture adapted isolate, growing and purifying the viral isolate, inactivating and or attenuating the virus and mixing an appropriate titer with a physiologically acceptable diluent and an immune stimulating agent. Alternatively, viral proteins can be purified for a subunit vaccine and an appropriate amount mixed with a physiologically acceptable diluent and an immune stimulating agent. The virus can be purified enough that there will be no contaminating material or substance that could interfere with the inactivation step and/or the immunogenicity of the virus.

An appropriate titer of virus or concentration of viral proteins can be admixed with the diluent and the immune stimulating agent. Measurement of TCID₅₀ is one way to measure virus titer (50% tissue culture infective dose). For example a titer of from about 10⁵ to 10¹² TCID₅₀ (based on pre-inactivation titers) can be used, including but not limited to 10⁶, 10^{7,} 10^{8,} 10^{9,} 10¹⁰ and 10¹¹. Optionally the titer can be analyzed by the HA titer and can contain from about 1 to 30 μg of HA per virus included in the vaccine, including but not limited to 1 to 10 μg for adjuvanted formulations and 1 to 30 μg for non-adjuvanted vaccines. In some embodiments, the titer is about 15 μg. Thus, for example, when 3 viruses are included in an unadjuvanted vaccine, 1 dose for adults contains the equivalent of 45 μg HA (15 μg for each of the 3 virus strains). In other embodiments, the amount from each strain may differ (for example depending on the antigenicity), but the final concentration is from about 1 to about 60 μg HA, including 2, 3, 5, 10, 15, 20, 25, 30, 35, 40, 45, 50 and 55 μg. In another embodiment, adjuvanted vaccines are expected to contain quantities of HA from about 1 to 30 μg, including 2, 5, 10, and 20 μg. The vaccine is typically of a volume between about 50 μl and 5000 μl, including 100, 500, 1000, 2000, and 5000 μls.

Standard physiologically acceptable diluents can be used, including, for example, EMEM, Hank's Balanced Salt Solution, and Phosphate Buffered Saline (PBS) and Normal Saline.

Appropriate immune-stimulating adjuvants can be added to the vaccine and/or pharmaceutical composition. Examples of immune-stimulating adjuvants, include, but are not limited to: mineral oil, vegetable oil, aluminum hydroxide, saponin, non-ionic detergents, squalene, block co-polymers, nano-beads, ISCOM, ISCOM matrix or other compounds known in the art, used alone or in combination.

In addition to adjuvant, any appropriate antiviral agents that are useful against influenza can also be included in a pharmaceutical composition. Such antiviral agents include, for example, rimantadine, amantadine, neuraminidase inhibitors (such as zanamivir and oseltamivir), gamma interferon, guanidine, hydroxybenzimidazole interferon alpha, interferon beta, thiosemicarbarzones, methisazone, rifampin, ribavirin, pyrimidine or purine analogs, and foscarnet.

Vaccines having more than one virus or strain of viral proteins can be produced using the methods herein. The mixtures can be immunogenically titrated to provide approximately equivalent immunogenicity. Immunogenically titrated means that the final product is produced to even out differences in immunogenicity. For example, if a mixture of strain A and strain B is prepared and strain A is 5 times more immunogenic, the strains are mixed at a ratio of strain A:strain B of 1:5.

### IV. Administration of Vaccine

The administration of the vaccine composition and/or the pharmaceutical composition can be for prophylactic purposes. When provided prophylactically, the compositions are provided before any symptoms of influenza viral infection is evident. The prophylactic administration of the composition serves to prevent or attenuate any subsequent infection. The pharmaceutical composition and/or vaccine can be administered in any way known in the art, including via inhalation, intranasally (for example with attenuated vaccines), orally, and parenterally. Examples of parental routes of administration include intradermal, intramuscular, intravenous, intraperitoneal and subcutaneous. In some embodiments, the vaccine is administered by intramuscular or deep subcutaneous injection in the upper arm. A second dose can be given after an interval of 2 to 4 weeks to some children who were not previously vaccinated for or exposed to influenza (unprimed). One or more booster vaccinations may be administered at an appropriate time after the initial immunization.

An effective amount of the vaccine and/or pharmaceutical composition is administered. An effective amount is an amount sufficient to achieve a desired biological effect such as to induce enough humoral or cellular immunity. This may be dependent upon the type of vaccine, the age, sex, health, and weight of the recipient. Examples of desired biological effects include, but are not limited to, production of no symptoms, reduction in symptoms, reduction in virus titer in tissues or nasal secretions, complete protection against infection by influenza virus, and partial protection against infection by influenza virus.

In some embodiments, an immunologically effective amount of a CIV vaccine is from about 100 HAU to about 1500 HAU per dose. The composition typically is between 250 and 750 HAU per dose. In one embodiment, the vaccine composition includes about 500 HAU per dose.

When administered as a solution, the vaccine can be prepared in the form of an aqueous solution, a syrup, an elixir, or a tincture. Such formulations are known the art, and are prepared by dissolution of the antigen and other appropriate additives in the appropriate solvent systems. Such solvents include water, saline, ethanol, ethylene glycol, glycerol, and A1 fluid, for example. Suitable additives include certified dyes, flavors, sweeteners, and antimicrobial preservatives, such as thimerosal (sodium ethylmercuithiosalicylate). Such solutions may be stabilized using standard methods, for example, by addition of partially hydrolyzed gelatin, sorbitol, or cell culture medium and may be buffered using standard methods, using, for example reagents such as sodium hydrogen phosphate, sodium dihydrogen, phosphate, potassium hydrogen phosphate and/or potassium dihydrogen phosphate. Liquid formulations may also include suspensions and emulsions. The preparation of suspensions, for example using a colloid mill, and emulsions for example using a homogenizer.

### V. Viral Strains and WHO

In order to give time for adequate vaccine stocks to be produced, a decision must be made well before flu season, as to which influenza A and B strains to use for this years vaccine (for the winter season). There is an elaborate and sophisticated epidemiological monitoring systems worldwide, which helps these decisions. In addition, the WHO usually prepares seed virus for use in producing vaccines.

There are 16 known HA subtypes and 9 known NA subtypes. Many different combinations of HA and NA proteins are possible. Only some influenza A subtypes (i.e., H1N1, H1N2 and H3N2) are currently in general circulation among people. Other subtypes are found most commonly in other animal species. For example. H7N7 and H3N8 viruses cause illness in horses, and H3N8 also has recently been shown to cause illness in dogs.

Three prominent subtypes of the avian influenza A viruses that are known to infect both birds and people are Influenza A H5-H5 infections, such as HPAI H5N1 viruses, Influenza A H7, and Influenza A H9. However, the next strains that infect humans and cause epidemics or pandemics could come from any subtypes.

Virus can be obtained using standard methods, for example, from patient samples, the American Type Culture Collection (or other collection) or from specific laboratories working on the virus in some embodiments, the virus is obtained from the WHO or CDC, including seasonal viruses and possible pandemic strains.

### VI. Detection of Serological Response

Methods of identifying whether the vaccine induces a serological response are also well known in the art. For example, one can inject a test animal with the immunogenic compound/vaccine and identify antiviral antibodies in the blood serum. Methods of identifying whether the vaccine is protective are well known in the art and include immunization of a test animal with the vaccine, followed by inoculation with a disease-causing virus and identification of the presence or absence of symptoms of the disease.

The Hemagglutination inhibition test can be performed to identify the presence of a serological response to hemagglutinin. The hemagglutination inhibition (HAI) assay may be performed with turkey red blood cells (RBCs) on all test serum samples, for example by using a known influenza subtype such as CIV(H3N8). Briefly, a serial two-fold dilution of test serum is performed in PBS in V-bottomed 96-well microliter plates. An equal volume of virus suspension containing 4-8 HAU/50 μl of CIV is added to each well containing test serum, and the plates incubated at room temperature for 30 minutes. Then, an equal volume of 0.5% turkey RBC suspension was added. The plates are then incubated at room temperature for 30 min and HAI results are read. The reciprocal of the highest dilution of the serum showing HA inhibition is considered as the HAI titer of the test sample.

Other methods of determining the presence of antibodies to influenza virus include the Neuraminidase inhibition test, Western blot, ELISA, PCR, and other methods for the identification of influenza virus antibodies. These assays are known in the art.

### VII. EXAMPLES

The following examples provide procedures for isolation, adaptation and purification of influenza virus to create a homologous virus population for the production of a master seed. The examples use Vero cells (American Type Culture Collection, CCL 81), but any cell type could be used that is permissive for influenza virus.

### Example 1

### Chemicals and Biologicals

The infection medium used contained 1 Liter DMEM (Cambrex, Catalog No. 04-096) or equivalent; stored at 2-7° C., 20 mL L-Glutamine (Cellgro, Catalog No. 25-005-CV) or equivalent; stored frozen at −10° C. or colder. Once thawed, it was stored at 2-7° C. for up to 4 weeks, and Type IX Trypsin (Sigma Product No. T0303, CAS No. 9002-07-7) or equivalent; was aliquoted and stored frozen at −5 to −30° C. The infection medium was freshly made before infecting the cells.

The cell culture medium preparation was as follows, 1 Liter DMEM, 20 mL L-Glutamine, 50 mL Fetal Bovine Serum (Gibco Catalog No. 04-4000DK) or equivalent (Note: Sourced from a BSE free country). The complete medium was stored at 2-7° C. for no more than 30 days after preparation.

EDTA-Trypsin (Cellgro Catalog No. 98-102-CV or equivalent) used for passaging the cells was stored at −5 to −30° C., expiration date was assigned by the manufacturer.

### Example 2

### Cell Culture Preparation

Because the dilution of protease to be used for the virus infection step can vary by lot, new trysin lots were titrated to establish the optimal level prior to use. An example of the titration is to serial dilute type IX trypsin in DMEM containing L-glutamine, using half log dilutions (10⁻¹, 10^{-1.5}, 10⁻², 10^{-2.5}, etc.). Using a 96 well plate containing a freshly confluent monolayer of Vero cells, wash each well of the plate 2 times using 280 μL of PBS. Immediately after washing, add 200 μL of each dilution of type IX trypsin to a row of the plate. The plate is then incubated at 37° C. plus or minus 2° C. with 5% CO₂ and the cells are observed after 4 days. The lowest dilution of trypsin that shows no or little effect on the health of the cells is selected as an appropriate concentration of trypsin to use for both isolation and optimization of infection with influenza. It is desirable and common to see little or no variation between wells inoculated within each concentration.

For limit dilution cloning of virus in the Vero cells, a confluent monolayer was used, typically 3-4 days of age; grown in 96 well Falcon microtest plates. The cells were derived from ATCC CCL 81 and used between passages 132 and 156.

Preparation of Vero cells from liquid nitrogen (LN₂) was as follows: one ampule of Vero cells was removed from LN₂ and thawed in a 36° C. plus or minus 2° C. water bath. The entire contents of the vial was pipetted into a 2 cm² tissue culture flask containing 10 mL of cell culture media that is supplemented with 10% fetal bovine serum. The flask was incubated at 36° C. plus or minus 2° C. in 4-6% CO₂. After about 1 hour, the supernatant and unattached cells were gently removed and 10 mL of fresh tissue culture media added. Cells were incubated at 36° C. plus or minus 2° C. in 4-6% CO₂ until 90-100% confluency was reached,

Passage of the Vero cells was as follows: Monolayers were washed using 10-20 mL of PBS for approximately 3 minutes. PBS was decanted and replaced with 3 mL of EDTA-trypsin (Cellgro, Catalog No. 98-102-CM), the monolayer was incubated for approximately 3 minutes or until the cells were detached from the flask. The suspension was diluted by adding 17 mL of prepared growth media (containing FBS) to dilute and neutralize the trypsin. Cells were then counted using a hemocytometer to determine the cell count per mL of suspension. The number of flasks that could be prepared using this suspension was calculated by: Cells per mL (suspension) X mLs of desired suspension mLs of plate for each vessel. Cells per mL (desired) Total the mLs of suspension, the total mLs of suspension must be less the volume available. If not the plating cell density can be adjusted to accommodate this, however it should be noted that the length of time until the cells are confluent will be longer with a lower cell density at plating. Vessels were usually plated using cells suspensions with a cell density of 1×10⁴ to 1×10⁵ cells per mL. Cells were incubated for 3-4 days, or until confluent.

These techniques for maintaining and propagating Vero cells were similarly applied to other cell lines used such as the Madin Darby Canine Kidney (MDCK) and HEK 293.

HEK 293 cells particularly suited for propagation of viral isolates was cloned. This HEK 293 subclone, designated GT-D22 (or D22), was isolated from an initial preparation of HEK 293 cells (ATCC No. CRL-1573; ATCC Batch No. F-11285 at Passage 33). HEK 293 cells were subcloned ad selected on the basis of improved productivity of recombinant adenovirus Type 5 carrying the gene for expression of p53. Clones having normal morphologies and adequate growth rates were trypsinized and seeded at 1-2×10⁶ cells per well for further analysis. Top producing clones were subjected to further subcloning and selection. The D22 subclone was finally selected. The ability of the D22 subclone to propagate Influenza has been demonstrated using Swine Influenza Virus (SIV).

Biosafety precautions were taken when working with live influenza virus. Influenza is a Class 2 etiologic agent and recommendations found in CDC-NIH HHS Publication No. (CDC) 88-8395 (Biosafety in Microbiological and Biomedical Laboratories) for handling the virus in the laboratory were followed.

### Example 3

### Limit Dilution Coning

Preparation of Dilution Tubes and Sample Dilution for Limit Dilution Cloning was as follows. Test tubes (12×75 mm) were set up in racks and labeled. 1 sample was run per plate and the dilution series for each sample was 10⁻¹ to 10⁻¹⁰ Dilution medium was dispensed in 1.8 mL amounts into each test tube using a serological pipette. The first tube was labeled with the virus identification. Several additional tubes were prepared for use as diluent controls and for replacement if any errors are made during dilution performance. Samples were vortexed for approximately 5 seconds, then the initial dilution was made by pipetting 200 μL of sample into the 10⁻¹ dilution tube. Serial dilutions were continued to 10⁻¹⁰. For each dilution, the sample was vortexed and the pipette tip was changed between dilutions.

Dilutions were transferred to cell plates as follows. Immediately prior to use, the medium was aseptically poured from the cell plates. Each well was rinsed using a 12-channel pipetter 2-3 times with 280 μL of sterile PBS. The plates were aseptically emptied, but they were not allowed to dry out. Plates were labeled with the virus identification, date, and dilution scheme. Each dilution was vortexed briefly prior to addition to the plate. Using a motorized Finnpipette or other appropriate pipetter with 1000 μL tips, 200 μL per well of sample was inoculated into a single row of the plate. The samples were loaded according to virus concentration. First, 2 rows of diluent controls were added to the plate followed by the highest dilution of virus (10⁻¹⁰) and continuing with the remainder of the samples. Samples were loaded in sequence from 10⁻¹⁰ through 10⁻¹.

### Example 4

### Virus Preparation

Harvesting of the virus and the CPE was evaluated as follows. Following the 4 day incubation period, cytopathic effect (CPE) was read by microscopic examination. The presence of cellular debris, appearance of dead cells and lack of viable cells were used to characterize CPE because they are typical for influenza virus. Occasionally nonspecific interference was observed in the initial virus dilutions, therefore it was important to examine several dilutions for CPE. When evaluating CPE it was important to determine the extent of CPE in the wells of the highest dilution exhibiting CPE. The highest levels of success was achieved by selecting the wells of the highest sample dilution that exhibited CPE, but of those wells, preference was given to the well or wells that showed the smallest extent of CPE. Once selected, the contents of the well were harvested by using a single channel 1000 μL pipette to aspirate the fluids. Usually the fluids were pipetted repeatedly to remove loosely attached cells from the well bottom and to help break up any clumps of cells or virus in the suspension. The harvested fluids were then used to perform an additional round, or rounds, of limiting dilution cloning or were sometimes used to inoculate a fresh monolayer for the production of a post cloning seed. It was typical to perform 2 to 3 rounds of limiting dilution cloning in immediate succession to produce a uniform population of virus. HA titer was analyzed at each step in the process and the results are shown in Table 1.

A/Indonesia/05/2005 (INDOH5N1), A/Vietnam/1203/04 (VNH5N1), A/New Caledonia/20199 (A/NC/20/99(R)), and A/Wisconsin/67/05 (A/Wis/67/05R) are reassortment influenza viruses provided by the CDC. For the reassortment viruses the genes encoding surface glycoproteins HA and NA are from the influenza strain (INDOH5N1, VNH5N1, A/New Caledonia/20/99 and A/Wis/67/05) while the remaining internal genes are from A/PR/8/34. Limiting dilution cloning was also applied to wildtype (without PR8 reassortment) influenza strains A/New Caledonia/20/99 (A/NC/20/99), A/Wisconsin/67/05 (A/Wis/67/05) and B/Malaysis/2506/04. The viruses were generated by using reverse genetic techniques and passaged in embryonated eggs. The egg materials were directly used for limit dilution cloning in Vero cells (A procedure for HA titration is shown in Example 5).

TABLE 1
HA Titer* before and after Limit Dilution Cloning (LDC)

| | | | After | After | | |
|---|---|---|---|---|---|---|
| | Original** | Before*** | 2nd | 3rd | Roller | Bio- |
| influenza strain | materials | LDC | LDC | LDC | bottle | reactor |
| IndoH5N1 | 20,480 | 160 | 1,280 | 2,560 | 5,120 | 7,680 |
| VNH5N1 | 2560 | <40 | 640 | 1,280 | 5,120 | 7,680 |
| A/NC/20/99 | 5,600 | 640 | 1,280 | — | — | — |
| A/NC/20/99 (R) | 10,240 | 1,280 | 1,280 | — | 2,560 | 10,240 |
| A/Wis/67/05 (H3) | 2,560 | 640 | 1,280 | — | — | — |
| A/Wis/67/05(H3R) | 7,680 | 640 | 1,280 | — | 2,560 | 5,120 |
| B/Malaysia/2506/ | 2,560 | <40 | 640 | — | — | 5,120 |
| 04 | | | | | | |

*HA titer is expressed as Unit/mL.
**HA titer in egg embryonic material provided by CDC.
***HA titer from Vero cells infected directly with egg embryonic material provided by CDC.

As seen in the above Table 1, a cell culture system can produce a virus titer as high as that from eggs. Additionally, virus strains which showed no detectable growth on Vero cells at the outset, VNH5N1 and B/Malaysia/2506/04 could be adapted to grow on Vero cells by limiting dilution cloning. Propagation of VNH5N1 and B/Malaysia/2506/04 on the amniotic membrane of eggs prior to limiting dilution cloning enhanced the adaptation of these viruses to growth on Vero cells.

Using SRID (Single Radial Immunodiffusion) to quantitate hemagglutinin, up to 132 μg-mL hemagglutinin protein was obtained for B/Malaysia/2506/04 in the concentrated Vero cell culture medium. The yield in μg of HA/mL of culture was 10× better than known published methods using Vero cells. Currently, a dose of human vaccine is equivalent to about 15 μg/strain of virus. Therefore about 8-9 doses can be obtained from one ml, of concentrated Vero cell culture medium.

### Example 5

### Passage of Influenza through Amniotic Membrane Enhances for Virus Able to Grow on Tissue Culture Cells

Influenza virus VNH5N1-PR8/CDC-RG was received from the CDC. This is a reassortant virus composed of the H5 and N1 genes from a Vietnam strain of avian H5N1 influenza virus on the PR8 virus backbone. This virus was expanded in 11 day old embryonated eggs inoculated by the allantoic cavity. Allantoic fluids were harvested 2-3 days after inoculation and had a virus yield of 2560 hemagglutinin units (HAU)/ml.

Allantoic fluids (˜200 μl) were diluted in 5 mL DMEM containing 1.25 μg/ml Type IX Trypsin and allowed to absorb to a confluent monolayer of Vero Cells (ATCC CCL No. 81 at passage 147) for 60 minutes at 36±2° C. Cultures were fed with 25 mL DMEM containing 1.25 μg/ml Type IX Trysin and incubated for 3 days and culture supernatants harvested. There was no detectable hemagglutinin activity in the harvested fluids.

Virus-containing allantoic fluids were diluted 1:10.000 and 100 μl inoculated into both the allantoic cavity and onto amnionic membranes of 11 day-old embryonated eggs. Eggs were incubated at approximately 39° C. for three days and allantoic and amniotic fluids harvested separately.

Vero cells (passage 132 to 152) were planted into 96-well plates at 1×10⁴ to 1×10⁵ cells/ml, 200 μl/well in DMEM containing 5% fetal bovine serum and incubated for 3-4 days (until confluent) at 36±2° C., 3-5% CO₂. VNH5N1 virus in allantoic or amniotic fluids was serially diluted 10⁻¹ to 10⁻¹⁰ in DMEM containing 1.25 μg/ml type IX trypsin. Media was removed from Vero cells, wells rinsed with 280 μl of phosphate buffered saline and then 200 μl of each dilution of virus was inoculated into 8 replicate wells. Plates were incubated for 4 days at 36±2° C. 3-5% CO₂. Amniotic fluids resulted in virus that grew to higher titers on Vero cells as evidenced by cytopathic effects up to the 10⁻⁹ dilution for the amnion source virus compared to 10⁻⁷ dilution for the allantoic source virus (see Table 2). Virus from one well at the highest dilution showing cytopathic effects was harvested and cloned by limiting dilution a second time. Again, the amnionic fluid sourced virus showed cytopathic effects at higher dilutions than seen for allantoic source virus (approximately 10 times more virus),

TABLE 2
Limiting Dilution Passage 1

| Allantoic Fluid Virus | | | | | | | | | Amnionic Fluid Virus | | | | | | | | |
|---|---|---|---|---|---|---|---|---|---|---|---|---|---|---|---|---|---|
| dilution | Cytopathic Effect (+ present) | | | | | | | | dilution | Cytopathic Effect (+ present) | | | | | | | |
| 10⁻¹⁰ | − | − | − | − | − | − | − | − | 10⁻¹⁰ | − | − | − | − | − | − | − | − |
| 10⁻⁹ | − | − | − | − | − | − | − | − | 10⁻⁹ | − | − | − | − | − | − | + | − |
| 10⁻⁸ | − | − | − | − | − | − | − | − | 10⁻⁸ | − | − | − | − | − | − | + | + |
| 10⁻⁷ | + | + | − | + | − | − | + | + | 10⁻⁷ | + | + | + | + | + | + | + | + |
| 10⁻⁶ | + | + | + | + | + | + | + | + | 10⁻⁶ | + | + | + | + | + | + | + | + |
| 10⁻⁵ | + | + | + | + | + | + | + | + | 10⁻⁵ | + | + | + | + | + | + | + | + |
| 10⁻⁴ | + | + | + | + | + | + | + | + | 10⁻⁴ | + | + | + | + | + | + | + | + |
| 10⁻³ | + | + | + | + | + | + | + | + | 10⁻³ | + | + | + | + | + | + | + | + |
| 10⁻² | + | + | + | + | + | + | + | + | 10⁻² | + | + | + | + | + | + | + | + |
| 10⁻¹ | + | + | + | + | + | + | + | + | 10⁻¹ | + | + | + | + | + | + | + | + |
| | A | B | C | D | E | F | G | H | | A | B | C | D | E | F | G | H |

| | |
|---|---|
| Virus from well H7 was harvested | Virus from well G9 was harvested |

Limiting Dilution Passage 2

| Allantoic H7 clone | | | | | | | | | Amnion G9 clone | | | | | | | | |
|---|---|---|---|---|---|---|---|---|---|---|---|---|---|---|---|---|---|
| dilution | Cytopathic Effect (+ present) | | | | | | | | dilution | Cytopathic Effect (+ present) | | | | | | | |
| 10⁻¹⁰ | − | − | − | − | − | − | − | − | 10⁻¹⁰ | − | − | − | − | − | − | − | − |
| 10⁻⁹ | − | − | − | − | − | − | − | − | 10⁻⁹ | − | − | − | − | − | − | − | − |
| 10⁻⁸ | − | − | − | − | − | − | − | − | 10⁻⁸ | − | − | − | − | − | − | − | − |
| 10⁻⁷ | − | − | − | − | − | − | − | − | 10⁻⁷ | − | − | − | − | − | − | − | − |
| 10⁻⁶ | − | − | − | − | − | − | − | − | 10⁻⁶ | − | − | − | − | − | − | − | − |
| 10⁻⁵ | − | − | − | − | − | − | − | − | 10⁻⁵ | − | − | − | − | − | − | − | − |
| 10⁻⁴ | − | − | − | − | − | + | − | − | 10⁻⁴ | + | + | + | + | + | + | + | + |
| 10⁻³ | + | + | + | + | + | + | + | + | 10⁻³ | + | + | + | + | + | + | + | + |
| 10⁻² | + | + | + | + | + | + | + | + | 10⁻² | + | + | + | + | + | + | + | + |
| 10⁻¹ | + | + | + | + | + | + | + | + | 10⁻¹ | + | + | + | + | + | + | + | + |
| | A | B | C | D | E | F | G | H | | A | B | C | D | E | F | G | H |

| | |
|---|---|
| Virus from well H3 was harvested | Virus from well G4 was harvested |

Limiting Dilution Passage 3

| Allantoic H7H3 clone | | | | | | | | | Amnion G9G4 clone | | | | | | | | |
|---|---|---|---|---|---|---|---|---|---|---|---|---|---|---|---|---|---|
| dilution | Cytopathic Effect (+ present) | | | | | | | | dilution | Cytopathic Effect (+ present) | | | | | | | |
| 10⁻¹⁰ | − | − | − | − | − | − | − | − | 10⁻¹⁰ | − | − | − | − | − | − | − | − |
| 10⁻⁹ | − | − | − | − | − | − | − | − | 10⁻⁹ | − | − | − | − | − | − | − | − |
| 10⁻⁸ | − | − | − | − | − | − | − | − | 10⁻⁸ | − | − | − | − | − | − | − | − |
| 10⁻⁷ | − | − | − | − | − | − | − | − | 10⁻⁷ | − | − | − | − | − | − | − | − |
| 10⁻⁶ | − | − | − | − | − | − | − | − | 10⁻⁶ | − | − | − | − | − | − | − | − |
| 10⁻⁵ | − | − | − | − | − | − | − | − | 10⁻⁵ | − | − | − | − | − | − | − | − |
| 10⁻⁴ | − | + | + | − | + | + | + | + | 10⁻⁴ | + | + | + | + | + | + | + | + |
| 10⁻³ | + | + | + | + | + | + | + | + | 10⁻³ | + | + | + | + | + | + | + | + |
| 10⁻² | + | + | + | + | + | + | + | + | 10⁻² | + | + | + | + | + | + | + | + |
| 10⁻¹ | + | + | + | + | + | + | + | + | 10⁻¹ | + | + | + | + | + | + | + | + |
| | A | B | C | D | E | F | G | H | | A | B | C | D | E | F | G | H |

| | |
|---|---|
| Virus from wells B4 and F4 was harvested | Virus from wells C5 & G5 was harvested |

Influenza clones, in Table 2, were identified based upon a combination of the column designation A-H and row 1-10 (dilution series 10⁻¹ through 10⁻¹⁰ (respectively). For example the clone name B4 represents virus harvested from the well found at column B and row 4 (10⁻⁴ dilution).

Virus harvested from the third limiting dilution (˜100 μl) were diluted in 5 ml of DMEM containing 1.25 μg/ml type IX trypsin and inoculated onto confluent Vero cells (passage 132-152) and incubated for 60 minutes at 36±2° C. After absorption, 45 ml of DMEM containing 1.25 μg/ml type IX Trypsin was added and cultures were incubated for four days at 36±2° C. Harvested culture supernatants were tested for hemagglutinin and both clones derived from amnion expanded virus resulted in four to eight times higher yields of HA (see Table 3).

TABLE 3

| Source virus | Clone Designation | Yield (HAU/ml) |
|---|---|---|
| Allantoic fluid | H7H3F4 | 320 |
| | H7H3B4 | 160 |
| Amniotic fluid | G9G4C5 | 1280 |
| | G9G4G5 | 1280 |

The G9G4C5 virus was subsequently expanded by growth in Vero cells in roller bottles (resulted in 5120 HAU/ml) and 5 liter bioreactors (resulted in 7680 HAU/ml).

Similar results were obtained with the Type B Influenza virus. B/Malaysia/2506/04. This virus also failed to yield any measurable hemagglutinin when virus obtained from allantoic fluid was propagated in Vero cells. However, virus derived from amnion expansion resulted in 640 HAU/ml after two limited dilution clonings and 5120 HAU/ml after expansion in a 5 liter bioreactor.

### Example 6

### Quantification of Hemagglutinin

The purpose of this procedure was to quantitate influenza virus hemagglutinin activity in viral fluids in the final product.

The materials used included: PBS. Cambrex, 517-16Q or equivalent, Alsevers Solution, E8085 or equivalent, fresh Rooster erythrocytes in Alsevers Solution (1:1 ratio). Allow to sit overnight in Alsevers to stabilize the receptors. Wash 2 times and store as a 10% suspension in PBS, or as a 50% suspension in Alsevers. Use within 4 days of collection, Microtiter plates, Falcon U-bottom plates, Catalog No. 3911 or equivalent, 8 channel micropipette, 5-50 μL, or equivalent, Centrifuge Beckman TJ-6, or equivalent, 20-200 μL micropipette or equivalent, Disposable 200 μL pipette tips. Positive Control Virus with known titer, inactivated antigen. Stored at 2-7° C. and used as is on the day of the test.

A. A standardized 0.5% Rooster Red Blood Cell (rRBC) suspension in PBS was prepared by first allowing the rRBC solution to equilibrate to room temperature (15-30° C.). Rooster RBC's in Alseveres has a 4 day expiration period from date of collection. A sufficient volume of rRBC's in Alsevers was transferred to a 50 mL conical centrifuge tube. The rRBC's were washed by filling, the tube to the 45 mL mark with PBS or Alsevers and mixed by inverting tube several times, then centrifuged at 400×g, at 4° C. for 10 minutes. The supernatant was removed with a pipette. If there was any hemolysis in the supernatant, the wash steps were repeated up to three times. After the final wash, 0.25 mL of packed rooster RBCs were added to 49.75 mL PBS and inverted to mix. The cell suspension was labeled with the date of preparation, PBS lot number used, and 0.5% Rooster Red Blood Cells in PBS, store at 2-7° C. Maximum storage time is 4 days).

B. The number of microtiter plates necessary to test the sample material was determined. All test samples were tested using two rows each of a 1:7 and 1:3 dilution scheme. Two rows of the positive control virus at a 1:2 dilution scheme and two rows as a PBS control were also used. Other samples were tested as requested. Using a black permanent marker the row designation on the microtiter plate was indicated. An example is shown below as Table 2.

TABLE 2
microtiter plate

| | | 1 | 2 | 3 | 4 | 5 | 6 | 7 | 8 | 9 | 10 | 11 | 12 |
|---|---|---|---|---|---|---|---|---|---|---|---|---|---|
| Serial No. | A | O | O | O | O | O | O | O | O | O | O | O | O |
| 1:2 | B | O | O | O | O | O | O | O | O | O | O | O | O |
| Dilution | | | | | | | | | | | | | |
| Serial No. | C | O | O | O | O | O | O | O | O | O | O | O | O |
| 1:3 | D | O | O | O | O | O | O | O | O | O | O | O | O |
| Dilution | | | | | | | | | | | | | |
| Positive | E | O | O | O | O | O | O | O | O | O | O | O | O |
| Control | F | O | O | O | O | O | O | O | O | O | O | O | O |
| Virus | | | | | | | | | | | | | |
| 1:2 | | | | | | | | | | | | | |
| Dilution | | | | | | | | | | | | | |
| PBS | G | O | O | O | O | O | O | O | O | O | O | O | O |
| PBS | H | O | O | O | O | O | O | O | O | O | O | O | O |

50 μL of PBS was added to each well of the microtiter plate. An additional 50 μL of PBS was added to well number one of those rows using a 1.3 dilution scheme and the PBS control rows. Each microliter plate from the point of sample addition up to the addition of rRBC's was completed before proceeding to the next microtiter plate. 50 μL of sample and positive control virus was added to well one of the designated rows. Serial two-fold dilutions of the samples were prepared using the multichannel pipettor, transferring 50 μL aliquots. The appropriate tips were aseptically pushed onto the multi-channel pipettor, assuring that the pipettor was set at 50 μL. The well contents of one column were mixed by drawing up and expelling the material a minimum of seven times. The tips used for mixing were discarded. More tips were pushed on and 50 μL of material in each well was transferred to the next column of wells. These steps were repeated until all rows were sequentially diluted. Removal of 50 μL from row twelve of the microtiter plates was assured, 50 μl of 0.5% it rRBC suspension was delivered to each well using the multi-channel pipettor. The rRBC suspension was added from the wells of the highest dilution to the lowest dilution. Each plate was agitated gently to mix the contents. A lid cover was placed over the top microtiter plate and the plates where stacked and incubated at room temperature (approximately 20-25° C.) for 45-60 minutes.

After the incubation period, the plates were set on a microtiter plate viewer to read and to determine whether the PBS control wells are acceptable (the PBS wells should not exhibit any hemagglutination). The characteristics were also determined. The rRBC's must have settled out as a complete button without any shield. A shield reaction is a dispersion of the rRBC's. If the PBS control wells were acceptable, the other wells were scored for hemagglutination. If the PBS control wells were not acceptable the test was invalid and was repeated. The hemagglutination results were recorded as positive (+) for hemagglutination, partial (+/−), and negative (0). A positive reaction showed a complete shield of the rRBC's or total dispersion of the cells. A negative reaction showed a total button formed by the rRBC's Wells that exhibited a “+/−” were considered to be negative for purposes of endpoint calculation. The highest dilution at which agglutination (no button) occurred was identified for each of the replicates of each dilution (i.e. the 1:2 and 1:3) and the titers were computed as the inverse of the last dilution demonstrating complete agglutination. The titer level of the samples and positive control virus tested were identified. The arithmetic mean of the endpoint titer of each set of duplicate dilutions was determined. The HA units per 0.5 mL (50 μL) of the Sample, PBS and Positive Control Virus were also determined. The HA units per 1 mL was calculated by multiplying the 0.05 mL value by 20.

The calculation was performed as follows: For each of the 1:2 and 1:3: test sample dilution, the arithmetic mean of the duplicates was recorded. The highest titer was recorded. HA/0.05 mL×20=HA/1 mL was multiplied. The Off Test Date and the Results as HA units per 1 mL (virus fluids) or HA units per dose (final product) were recorded. A valid test for bulks or final products contains complete agglutination (no button) at the lowest dilution and no agglutination (button) at the highest dilution. The positive control lot should be within the established range. A titer range outside the listed parameters constitutes a “no test” or an invalid test and should be repeated without bias.

### Example 7

### Production of a Vaccine

The virus strain is a pandemic strain or seasonal strains designated by the WHO, CDC, or other governmental organizations. For the purposes of validation of the human vaccine manufacturing process, the influenza virus reassortant VNH5N1-PR8/CDC-RG reference strain provided by CDC is used. Phosphate buffered saline is used as a diluent for the vaccine preparation and ISCOM as the adjuvant. A lipid mixture of equal parts of cholesterol and phosphatidyl choline is used to assist the hydrophilic/hydrophobic complexing process in the ISCOM formation. The non-ionic detergent which is used to disrupt the virus is removed by diafiltration. Formation of ISCOMS is verified by electron microscopy. Binary ethylenimine (BEI) is used to inactivate the virus and then the BEI is neutralized with sodium thiosulfate. The process is set out in more detail in steps 1-19 below.

The influenza strain is produced in Vero (African Green Monkey Kidney) cells, inactivated with the aziridine compound binary ethyleneimine (BET), concentrated, purified and purified by filtration and gel chromatography. The virus is formulated with an adjuvant with Quil A and a lipid mixture to make the drug product.

Stage 1A—Vero cells are revived from the Working Cell Bank (WCB). Passage number is limited to 20 passages from Master Cell Bank (MCB). One ampule is thawed from WCB in liquid nitrogen and seeded at 4-5×10⁴ cells/cm² in Dulbecco's Modified Minimum Essential medium (DMEM) containing 20% v/v irradiated fetal bovine serum of New Zealand or Australian origin, 4 mM L-glutamine in (typically) a 25 cm² Nunc flask. The flask is incubated at 36° C. plus or minus 2° C., the supernatant and unattached cells are removed after about 1 hour, and the flask is refed with fresh media and incubated as before.

Stage 2A—Cell expansion is continued by harvesting of confluent monolayers using a Trypsin/EDTA solution, and re-planting cells into more static flasks or roller bottles. Further expansion may be performed in bioreactors by the use of microcarriers at 20-30 g/L.

Stage 3A—When the desired production volume of culture substrate is achieved in either the bioreactor or in roller bottles, cells are washed twice with Dulbecco's Modified Minimum Essential medium (DMEM) to remove residual serum, that will inactivate trypsin in the infection medium.

Stage 1B—Working Virus Seed (WVS) is prepared separately and frozen in advance of large-scale manufacturing. Master Seed Virus or Working Seed Virus (MSV+1) stored at −70° C. is thawed and diluted in virus infection medium containing Type IX porcine trypsin to achieve the desired MOI. A predetermined volume is inoculated onto confluent Vero cell monolayer in the roller bottles or bioreactor and incubated at 36° C. plus or minus 2.0° C., typically for 40-72 hours, when up to 100% cytopathetic effect (CPE) is identified. Virus is harvested and frozen at −50° C. or colder.

Stage 4—Working seed virus (not higher than passage MSV+2) is thawed and diluted in Virus Infection Medium containing 0.5-5.0 μg mL of Type IX porcine trypsin to achieve the desired multiplicity of infection. The use of trypsin in the medium will assist the attachment and penetration of the virus into the cell.

Stage 4A—production of influenza virus using a bioreactor. A 5-liter bioreactor was prepared with SoloHill Plastic Plus microcarriers at a density of 30 g/L. The bioreactor was planted at 2×10⁵ Vero cells/mL in Dulbecco's Modified Minimum Essential medium with 5% v/v irradiated fetal bovine serum of New Zealand or Australian origin, 4 mM L-glutamine and incubated at 36° C. plus or minus 2° C. After the cell confluency reached 80-100%, the microcarriers containing the Vero cells was settled and washed twice with 2 liters per wash of serum-free DMEM. Infection medium containing 2.5 μg/mL of trypsin IX was added to the bioreactor. Virus seed, for example VNH5N1, also was added to the bioreactor at a MOI of 0.0001-0.0003. The preparation of virus was continued for 5 days. The bioreactor was sampled daily an for CPE observation and HA titration. The virus was harvested after 80-100% CPE was reached.

Stage 5—Binary ethyleneimine (BEI) is added to the harvested virus to give a final concentration of 1.5 mM, and held at 36° C. plus or minus 2° C. for 1 hour (with agitation) at a pH of 7.3 plus or minus 0.3.

Stage 6—After stage 5 is complete, the harvest is transferred to a second vessel and the inactivation process continued at 36° C.±2° C. for 48 hours (with agitation). After this time sodium thiosulfate is added to a final concentration of 3 mM to neutralize any residual BEI.

Stage 7—The culture is clarified through 7μ and 1μ filters and stored at 2° C.-8° C. pending innocuity clearance. The innocuity test takes ten days to perform.

Stage 8—Antigen is concentrated using a tangential flow ultra filtration system, using a polysulfone membrane with a molecular weight cut off (MWCO) of 100 K. Up to approximately a 50 fold concentrate is achieved.

Stage 9—The resulting concentrated culture fluids are equilibrated in an appropriate buffer and treated with DNase (Benzonase) to degrade cellular DNA.

Stage 10—The concentrate is purified using size exclusion gel chromatography. The gel currently used is a cross linked Sepharose (CL-2B, Pharmacia). The column is 90 cm in length to achieve the required separation. CL-2B is a ‘soft’ get which depends on the wall of the column for support. Typically the 90 cm length is achieved by 2×45 cm or 3×30 cm columns (e.g., 30-32 cm in height by 30 cm in diameter) in series. The concentrated virus is applied at approximately 5-7% of the column volume.

Stage 11—Virus peak material is reconcentrated using a bench scale tangential flow ultra filtration system with a 100 K MWCO polysulfone membrane.

Stage 12—The re-concentrated virus peak material is solubilised by adding 5 ml, of a 10% (w/v) detergent (Nonanoyl-N-Methylglucamide) Mega 9 solution to 200 mls. of an antigen solution. The solution is mixed slowly with a magnetic stirring bar for 1 hour at 20-25° C. in a glass container.

Stage 13—A lipid mixture is added at 50 μl per 20 mLs of re-concentrated virus peak. The lipid mixture contains 10 mg/mL each of egg derived phosphatidylcholine and cholesterol. Stirring is continued at 20-25° C. to ensure even distribution of the lipids in the re-concentrated virus. Quil A (from 10% w/v stock solution) is added to give a final concentration of 0.05%. The solution is stirred for approximately 30 minutes at 20-25° C.

Stage 14—The Mega 9 detergent is removed from this mixture (to allow ISCOM formation) by diafiltration with 50 mM ammonium acetate. This is performed using the bench scale tangential flow ultrafiltration system with a 100 K MWCO polysulfone membrane. The volume of ammonium acetate used is a minimum of about 10× the volume of re-concentrated virus peak mixture. Diafiltration is effected by maintaining a constant volume throughout, by balancing feed and permeate flow. Detergent interferes with the formation of ISCOM. Electron microscopy verifies that the typical cage-like structures have been formed.

Stage 15—The ISCOM is re-concentrated as the final step of diafiltration.

Stage 16—After satisfactory QC release, batches of ISCOMs are formulated to make the vaccine at 1 to 20 μg human influenza HA per 1 mL dose. Phosphate buffered saline (PBS) is used as the diluent.

Stage 17—The blended vaccine is filled into single dose final containers under Class A conditions. Samples are taken for sterility, safety in lab animal species, extractable volume and visual appearance. Vaccine is labeled and packed and held in quarantine at 2° to 7° C.

Stage 18—After final QA clearance, product is released to finished goods' cold store (2° to 7° C.) pending dispatch.

### Example 8

### Efficacy of Vero Cell Derived Vaccine in Ferrets

The ability of Vero derived influenza vaccine to seroconvert ferrets was evaluated. A trivalent human influenza vaccine based on the 2006-2007 seasonal influenza strains (A/NC/20/99, A/Wis/67/05 both PR8 reassortments and B/Malaysia, all obtained from the CDC) was produced in Vero cells after limit dilution cloning. The resultant vaccine, “SPflu0607,” with or without ISCOM adjuvant, was injected into the left hind leg of 4-6 month old female ferrets. As control comparisons, commercially available human influenza vaccines Fluzone® (manufactured by Sanofi-Pasteur) and Fluvirin® (manufactured by Chiron) were tested along side SPflu0607. Single Radial Immune Diffusion (SRID) was used to measure the quantity of Hemagglutinin (HA) protein in the vaccine. Seroconversion of the ferrets was measured by hemagglutinin inhibition (HI). HI titers of greater or equal to 1:40 are considered positive. See Table 3

TABLE 3

| | Days after vaccination | | | | | | | |
|---|---|---|---|---|---|---|---|---|
| | 14 days | | 28 days | | 60 days | | 90 days | |
| Vaccine | GMT | % Positive | GMT | % Positive | GMT | % Positive | GMT | % Positive |
| SPflu0607 + ISCOM | | | | | | | | |
| 7.5 μg/mL | 403 | 100 | 118 | 89 | 40 | 56 | 29 | 56 |
| 3.8 μg/mL | 1186 | 100 | 254 | 100 | 127 | 100 | 54 | 67 |
| 1.9 μg/mL | 274 | 100 | 93 | 89 | 50 | 67 | 25 | 44 |
| SPflu0607 | | | | | | | | |
| 15 μg/mL | 118 | 67 | 43 | 67 | 29 | 56 | 20 | 33 |
| 7.5 μg/mL | 137 | 89 | 52 | 78 | 23 | 44 | 16 | 44 |
| Chiron | | | | | | | | |
| 15 μg/mL | 131 | 89 | 66 | 78 | 27 | 56 | 16 | 33 |
| 7.5 μg/mL | 148 | 78 | 63 | 67 | 32 | 56 | 29 | 56 |
| Sanofi | | | | | | | | |
| 15 μg/mL | 101 | 89 | 101 | 89 | 25 | 44 | 15 | 22 |
| 7.5 μg/mL | 135 | 88 | 80 | 88 | 28 | 50 | 12 | 25 |
| PBS Only | 7 | 0 | 6 | 0 | 6 | 0 | 5 | 0 |

GMT = Geometric mean antibody titer
% Positive = Percentage of animals with HI antibody levels ≧1:40

The above results indicate that Vero cell-derived vaccine is comparable to commercially available egg derived vaccines.

### Example 9

### Method of Canine Influenza Virus Propagation for Preparing a CIV Vaccine

Canine influenza was isolated from nasal secretions of a sick dog. Nasal swabs were taken and placed in 2 mL of tissue culture media containing gentamicin and amphotericin. 0.8 mL of the resultant swab material was inoculated onto confluent Madin Darby Canine Kidney (MIDCK) cells in 10 mL of DIMEM tissue culture media containing 1.3 μg mL type IX Trypsin and incubated for 2 days at 36±2° C. The flask was harvested by decanting the media and the virus was identified as H3N8 by the National Veterinary Services laboratory using standard anti-sera. The MDCK passaged virus contained 160 hemagglutinin units per ml. The virus was cloned by inoculating 10-fold serial dilutions onto confluent MDCK cells in 96-well plates and harvesting a singe well at the highest dilution showing cytopathic effects (tissue culture media was DMEM containing 1.3 μg/mL type IX trypsin). This procedure was repeated a second time. The clone was then expanded on MDCK cells in a 75 cm² flask. The resultant virus (passage 4) yielded 640 hemagglutinin units per ml. The virus was then passaged onto Madin Darby Bovine Kidney cells by inoculating 0.23 MOI onto a confluent monolayer in a 1050 cm² roller bottle using 300 mL DMEM containing 0.8 μg/mL type IX trypsin. The roller bottle was incubated for 3 days at 36±2° C. The harvested virus yielded 2560 HAU/ml. Due to the increased yield of HA on MDBK cells, this cell line was chosen for scale-up of virus for vaccine preparation. The virus was propagated in a bioreactor. A 5 L bioreactor was seeded with MDBK cells at 3.0×10⁵ cells/mL attached to Cytodex III microcarriers at 5 grams per liter. Cells were grown for 4 days in DMEM containing 5% fetal bovine serum with no antibiotics at 36±2° C. After settling the microcarriers, 90% of the media was removed and replaced with serum free DMEM. Type IX trypsin was added to a concentration of 10 μg/mL in the final 5,000 mL. The cells were infected with virus at an MOI of 0.01. The virus was incubated with the MDBK cells on microcarriers for 2 days at 36±2° C. and then the supernatant was harvested. The resulting virus yielded 10,240 HAU/ml.

### Example 10

### Limiting Dilution Cloning of Clinical Isolate without Passaging in Eggs Creates a Uniform Population and Improves TCID50/mL and HA Titer

Canine Influenza Virus H3N8 (wild type) was received from a diagnostic laboratory and isolated from a nasal swab. Upon receipt the nasal swab was processed and used to inoculate a 25 cm² flask containing a confluent monolayer of MDCK cells. Infection media was comprised of: DMEM, 4 mM L-glutamine/ml, 1.3 μg/ml Type IX, and gentamicin. The flask was incubated at 36±2° C. with 3-5% CO₂ and harvested at onset of CPE. An HA assay was preformed on the harvest fluids with a result of 160 HAU/ml and a TCID₅₀/ml titer of 7.94.

MDCK cells were planted into 96-well plates at 1×10⁴ to 1×10⁵ cells/ml, 200 μl/well in DMEM containing 5% fetal bovine serum and incubated for 3-4 days (until confluent) at 36±2° C., 3-5% CO₂. CIV H3N8 virus was diluted as specified in the section: Limiting Dilution Round 1. Dilutions were performed in DMEM containing 1.3 μg/ml type IX trypsin, 4 mM L-glutamine, and gentamicin. Media was removed from MDCK cells, wells rinsed with 280 μl of phosphate buffered saline and then 200 μl of each dilution of virus was inoculated into 8 replicate wells. Plates were incubated for 4 days at 36±2° C., 3-5% CO₂. Two rounds of limiting dilution cloning were preformed, limiting dilution round 2 immediately following limiting dilution round 1. The process yielded a virus isolate that produced both higher TCID₅₀/ml and hemagglutination titers. Virus from a well at the higher dilutions demonstrating the lowest extent of cytopathic effect was harvested and cloned by limiting dilution a second time.

### Limiting Dilution Round 1

Pass 1 material was titrated with a result of 7.5 TCID₅₀/ml. Using this value the virus was diluted to yield 5 samples.

A. 10⁻⁴

B. 10⁻⁵

C 10 virus particles/well (10^{'6.6})

D. 3 virus particles/well (10^{'7.02})

E. 1 virus particles/well (10^{'7.5})

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | |
|---|---|---|---|---|---|---|
| A | Diluent | 10⁻⁴ | 10⁻⁵ | 10^{-6.5} | 10^{-7.02} | 10^{-7.5} |
| B | Control | | | | | |
| C | | | | | | |
| D | | | | | | |
| E | | | | | | |
| F | | | | | | |
| G | | | | | | |
| H | | | | | | |

Well A11 was harvested in preparation for round 2 of limiting dilution cloning.

### Limiting Dilution Round 2

| | 1 | 2 | 3 | 4 | 5 | 6 | 7 | 8 | 9 | 10 | 11 | 12 |
|---|---|---|---|---|---|---|---|---|---|---|---|---|
| A | Diluent | 10-10 | 10-9 | 10-8 | 10-7 | 10-6 | 10-5 | 10-4 | 10-3 | 10-2 | 10-1 | Diluent |
| B | Control | | | | | | | | | | | Control |
| C | | | | | | | | | | | | |
| D | | | | | | | | | | | | |
| E | | | | | | | | | | | | |
| F | | | | | | | | | | | | |
| G | | | | | | | | | | | | |
| H | | | | | | | | | | | | |

Virus from Well A5 was Harvested

Virus harvested from the second round of limiting dilution cloning (˜200 μl) was used to inoculate a 75 cm² flask containing a confluent monolayer of MDCK cells and DMEM supplemented with; 1.3 μg/ml type IX trypsin, 4 mM L-glutamine/ml, and 25 μg/ml gentamicin. Harvested culture supernatants were titrated to determine the TCID₅₀/ml and hemagglutination titer (see Table 4).

TABLE 4

| Virus Passage | TCID₅₀/ml | HAU/ml |
|---|---|---|
| Pass 1 from field isolate | 7.94 | 160 HAU/ml |
| Pass 4, Pre-master seed | 7.69 | 640 HAU/mL |

In laboratory experiments performed for immunogenicity trials the virus was grown on MDBK cells in a 5 L bioreactor with a resulting hemagglutination titer of 10,240 HAU/ml.

### Example 11

### Efficacy of an Inactivated Canine Influenza Vaccine in Dogs

Canine influenza virus (CIV) serotype H3N8 causes severe respiratory disease in dogs. However, there is no effective vaccine against CIV currently available. The objective of this study is to evaluate the efficacy of an inactivated CIV vaccine, prepared by limiting dilution and propagation of CIV in tissue culture cells in preventing clinical disease and lung lesions induced by a virulent CIV challenge. The vaccine consists of binary ethyleneimine (BEI)-inactivated CIV antigen adjuvanted with Emunade® adjuvant with an antigen input level of 500 hemagglutination units (HAU) per dose. A group of eight 7-week-old CIV seronegative dogs was vaccinated intramuscularly with the vaccine and booster dose was administered 21 days after the primary vaccine. Two weeks following booster vaccination the vaccinated dogs exhibited significantly higher levels of HA inhibiting antibody titer compared to their non-vaccinated counterparts demonstrating stimulation of immune response by the vaccine in dogs. The non-vaccinated control and the vaccinated dogs were challenged with a heterologous virulent CIV isolate 16 days post-booster vaccination, and monitored daily for 10 days post-challenge for clinical signs, rectal temperature and nasal CIV shedding. All control dogs (100%) developed clinical signs including ocular and nasal discharge, sneezing and coughing indicating virulence of challenge virus. The vaccinated group exhibited significantly lower clinical signs (median score=4.3) compared to control group (median score=6.8; p=0.0051). Only one of the dogs in the vaccinated group (12.5%) showed nasal CIV shedding and it only was for one day, whereas, all the dogs in the control group (100%) had significantly higher virus shedding compared to the vaccinates (p=0.0003). The virus shedding lasted for 7 days following challenge in the control group. All dogs were euthanized 10 days after challenge, and necropsy was performed for evaluation of lung lesions. All dogs in the control group (100%) showed varying degrees of lung consolidation, whereas, only one in the vaccinated group (12.5%) exhibited a mild lung consolidation. The lung scores were significantly higher in control dogs (median score=4.9) when compared to vaccinated dogs (median score=0; p=0.0005). These results unequivocally demonstrate that the vaccine formulation tested in this study protects dogs against CIV infection by significantly decreasing clinical signs, reducing virus shedding, and preventing CIV-induced lung consolidation.

### Study Overview

The objective of the study was to test the efficacy of Emunade®-adjuvated CIV vaccine formulation to protect against CIV challenge in dogs.

Dogs were initially acclimatized for eight days. For the test group, the first vaccination occurred on day 0 and a booster was given on day 21. The control group was not vaccinated. Challenge with CIV was presented to both groups on day 37. The dogs were monitored and observed throughout the study, as described below. The dogs were euthanized 10 days after challenge and a necropsy was performed.

### Test Animals

Test animals were an average of 48.25 days old on day 0. There were 8 dogs in the control group and 8 dogs in the test group Average weight was 1.8 kg. The dogs used in the study did not have a history of respiratory infection or CIV vaccination. To confirm that the dogs were negative for CIV antibodies (HA titer<10), blood samples were collected on day −1, and tested by a hemagglutination inhibition assay. Nasal swabs were also taken on day −1 to ensure that the dogs were free of CIV at the time of vaccination.

### Pre-Vaccination Monitoring

Blood samples were collected from alt dogs in evacuated serum separation tubes on the day before administration of the first vaccine in order to confirm that the dogs were negative for CIV antibodies. Nasal swabs were taken on the same day to confirm that the dogs were free of CIV.

The general health of the dogs was assessed by physical examination of the dogs two days prior to first vaccination. Clinical assessments and rectal temperature were performed from two days before the initial vaccination and booster vaccination through the day of vaccination.

### Vaccination

Canine influenza virus vaccine CIV H3N8-Emunade® was used in this study. Canine Influenza Virus (H1-3N8) was initially isolated from a dog with severe respiratory disease. Madin-Darby Bovine Kidney (MDBK)-KC cells at MCS+19 passage level, i.e., following 19 passages of the Master Cell Stock, were used to propagate the CIV H3N8. The virus then was inactivated with 6 mM BEI for 60 hours at 36° C. The BEI was neutralized with 60 mM Sodium thiosulfate.

The vaccine for this study was prepared in an 800 mL stock for aliquoting into 800 one mL doses. The 800 mL solution was prepared as shown in Table 5.

TABLE 5

| Component | Volume Used | |
|---|---|---|
| Aqueous Phase: | | |
| Inactivated CIV | 156 | mL |
| (2560 HAU/mL) | | |
| Saline | 342 | mL |
| Aluminum hydroxide (2.1% Aluminum | 77 | mL |
| oxide) | | |
| Ethyl Alcohol | 16 | mL |
| Glycerin | 40 | mL |
| HEPES 1M | 8 | mL |
| 1% Red Dye Solution | 0.8 | mL |
| Oil Phase: | | |
| Mineral Oil | 107 | mL |
| Tween 80 | 34.5 | mL |
| Span 80 | 18.4 | mL |
| Preservative: | | |
| Gentamicin | 0.373 | mL |
| Total Volume | 800.0 | mL |

The inactivated CIV H3N8 virus was diluted in normal saline and then adjuvanted with aluminum hydroxide. The remaining components in aqueous phase were added to the adjuvanted antigen. The oil phase was prepared separately and then added to the aqueous phase over a period of 10 minutes with continuous mixing for one hour, The serial was homogenized using Silverson homogenizer for 30 minutes.

The CIV vaccine vials that were stored at 2-7° C. were equilibrated to room temperature for a minimum of 30 minutes. The vaccine was loaded into 3 mL syringes (1 mL per syringe) and used for immunization. The first dose of vaccine was administered intramuscularly into right hind leg on day 0 and the second dose was administered intramuscularly into left hind leg on day 21.

### Post Vaccination Procedures

Complete clinical assessments including rectal temperatures and injection site observation were performed on all dogs within 3-6 hours after each vaccination to measure any immediate reactions. The clinical assessments were continued daily for 7 days following each vaccination and scored according to the Clinical Assessment Guide below. Dogs were bled on study days 20 and 36, and the serum samples were used to measure CIV antibodies by hemagglutination inhibition.

### Pre Challenge Procedures

Clinical assessments were performed and rectal temperatures were recorded for all dogs for two days prior to challenge (days 35 and 36) and on the day of challenge (day 37) before challenge administration. The clinical signs were scored according to the Clinical Assessment Guide.

### Challenge

Challenge material: The CIV14-06A virus isolated from MDCK cells and used to challenge vaccinated does, was originally isolated from field sample collected from dog suffering with canine respiratory disease. The average titer of the challenge virus was 7.7 Log₁₀TCID₅₀/mL. On the day of challenge, the challenge material was diluted to 1:4 in sterile cold Dulbecco's Minimum Essential Medium (DMEM) to target a challenge dose of 7.4 Log₁₀TCID₅₀ per dog.

Challenge administration: All dogs were challenge-administered on day 37. Four dogs were placed in a Plexiglas chamber and 8 mL of challenge virus (2 mL/dog) was used to venerate aerosol over a period of approximately 20 minutes. The dogs were exposed to aerosol for a total of 40 minutes.

### Post-Challenge Monitoring

Rectal temperature was recorded and clinical assessments were performed for each dog daily for 10 days post-challenge. Nasal swabs were collected daily from each dog for 10 days post-challenge. Nasal swabs were processed and titrated daily after each collection as described herein. Blood samples were collected in evacuated serum separation tubes on day 10 post-challenge just before euthanasia.

### Necropsy

All challenged dogs were euthanized on day 10 post-challenge (Day 47) using an AVMA approved method (Ketamine cocktails and Beuthanasia-D), and necropsy was performed. Immediately following euthanasia, the lungs were evaluated. Areas of visible consolidation were assessed and scored as a percent consolidation of each lung lobe. The percentages were converted to weighted scores and total score for each dog was calculated. During necropsy, the lung tissue was collected for virus isolation and titration, and for histopathology.

### Virus Titration

Hemagglutination (HA) assay was performed for virus titer. The virus was serially two-fold diluted in a V-bottomed microtiter plate and an equal volume of 0.5°, turkey red blood cell (RBC) suspension was added to the virus suspension. The plates were incubated at room temperature for 30 min and HA results read. The highest dilution of the virus showing HA activity was considered as 1 HA unit. All assays were performed in duplicate and the endpoint HA titer was measured.

To confirm the potency of challenge material and to measure virus shedding in challenge-administered dogs, the virus titer in challenge material, nasal swab and lung tissue was determined by titration in MDCK cells. MDCK cells were seeded in 96-well tissue culture plates for two) days, and were inoculated with ten-fold serially diluted virus suspension or samples prepared from stung tissue and nasal swabs. The plates were incubated at 36±2° C. temperature and 5% CO₂. Seven days post-infection, the plates were observed for cytopathic effect (CPE), and the 50% end-point for infectivity was calculated using Spearman-Karber method. The virus titers were expressed as Log₁₀TCID₅₀/mL.

### Detection of Serological Response

Antibodies to CIV in dog serum samples were measured by hemagglutination inhibition (HAI) assay. Briefly, a serial two-fold dilution of test serum was performed in PBS in V-bottomed 96-well microtiter plates. An equal volume of virus suspension containing 4-8 HAU of CIV25-06B was added to each well containing test serum, and the plates were incubated at room temperature for 30 min for antigen-antibody reaction to occur. Then, an equal volume of 0.5% turkey RBC suspension was added. The plates were incubated at room temperature for 30 min and HAI results were read. The reciprocal of the highest dilution of the serum showing HA inhibition was considered as the HAI titer of the test sample. All assays were performed in duplicate and the endpoint HAI titer was determined.

### Results: Clinical Scores

All dogs were monitored daily, from two days prior to challenge through 10 days post-challenge, for clinical signs including ocular discharge, nasal discharge, sneezing, coughing, dyspnea and depression. Daily clinical scores for ocular discharge, nasal discharge, sneezing, coughing, depression and dyspnea for 10 days post-challenge were summed to obtain a summed clinical score for each dog. Summed clinical scores for vaccinated and control groups were compared using Wilcoxon Exact Rank Sum tests and two sided p-values were calculated.

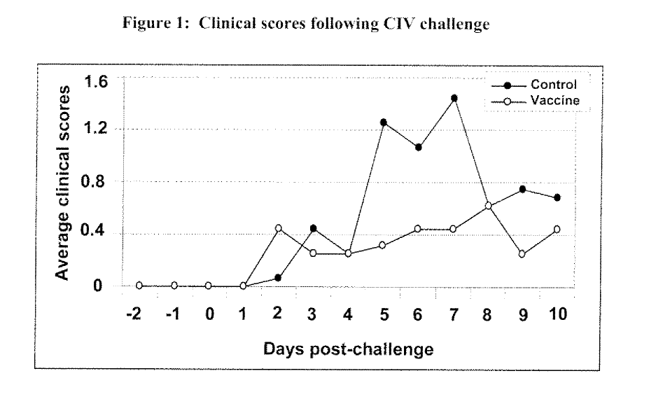

Dogs in both control and vaccinated groups exhibited a range of clinical signs starting from two days post-challenge (FIG. 1). All eight dogs (100%) in the control group exhibited varying degrees of coughing that lasted up to five days within 10-day post-challenge observation period. On the other hand, only two dogs (25%) in the vaccinated group exhibited a mild cough that was observed only one day during the entire 10-day post-challenge period. Coughing was the predominant sign exhibited by dogs in control group. On the contrary, only mild ocular discharge was the predominant clinical sign exhibited by vaccinated dogs. The clinical scores were significantly higher (p=0.0051) in control group (median score=6.8) compared to vaccinated dogs (median score=4.3). These data suggest that the CIV vaccine protects dogs against CIV-induced clinical signs.

### Results: Nasal Virus Shedding

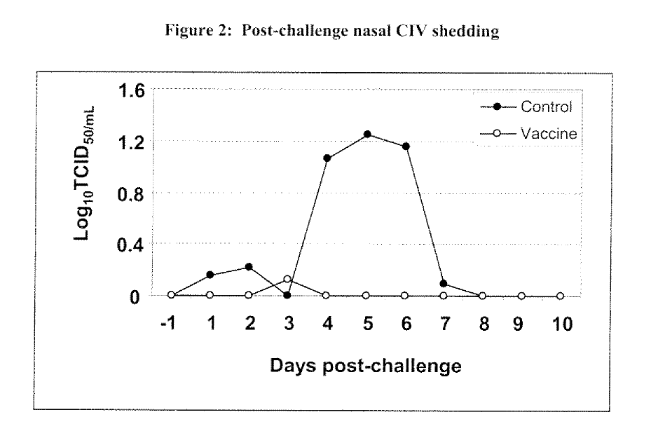

Nasal virus shedding was monitored in all dogs by collecting and processing nasal swabs on the day before challenge (day-1), and then daily from day 1 through day 10 post-challenge. The virus titers (Log₁₀TCID₅₀/mL) of the nasal swabs were determined and plotted against time. Area under the curve was compared between control and vaccinated groups using Wilcoxon Exact Rank Sum tests. The average virus titer for each groups expressed as Log₁₀TCID₅₀/mL, was plotted against days post-challenge (FIG. 2).

The control group started shedding virus in nasal secretions from day 1 post-challenge. The virus shedding reached its peak on day 5 post-challenge (1.25 Log₁₀TCID₅₀/mL) followed by a precipitous drop on day 7 (FIG. 2). All dogs in control group (100%) were positive for virus shedding at one or moor time points during 10-day post-challenge observation. On the other hand, only one dog (12.5%) in vaccinated group (ID No CXTAMM) shed virus in nasal secretions only for one day (day 3). Non-vaccinated control dogs exhibited significantly higher nasal virus shedding compared to vaccinated dogs (p=0.0003). These results clearly indicate that the CIV vaccine significantly inhibits nasal virus shedding by vaccinated dogs.

### Results: Serological Response

Geometric mean antibody titers (GMT) from HAI assays were calculated following primary and booster vaccinations. The fold increase in titers between immunizations was reported. The antibody titers were compared between control and vaccinated groups using Wilcoxon Exact Rank Suns tests. All 16 dogs enrolled in the study were healthy and seronegative (i.e., negative for CIV antibodies) (HAI titer of <10) at the time of primary vaccination. Nasal swabs collected on the day before vaccination (Day −1) confirmed that the dogs were free of nasal CIV shedding. Control dogs remained seronegative at the time of challenge.

The HAI antibody titers were tabulated and compared between control and vaccinated groups. All vaccinated does venerated measurable levels of antibody titers following first vaccination. The HAI antibody titters ranged between 10 and 40 with a GMT of 22 and the titers were significant when compared to control dogs (p=0.0070). Second vaccination boosted the antibody titers by six fold (GMT=135) which were significantly higher than control (p=0.0002). The antibody titers ranged between 80 and 160, with most of the dogs exhibiting an HAI titer of 160 (75%). All the control dogs remained free of CIV antibodies until the time of challenge (HAI titer<10). Following challenge, antibody titers in vaccinated dogs reached very high levels (GMT=546) demonstrating efficacy of the vaccine in priming the immune system against virulent CIV. The HAI titers in these dogs ranged between 120 and 1920. The non-vaccinated controls also made antibodies following CIV challenge with GMT of 149.

### Results: Lung Consolidation, Virus Isolation, and Histopathology

Lung consolidation pneumonia is the major pathological lesion in all influenza infection. In the previous study on development of challenge model, we observed severe lung consolidation in dogs on 6 and 14 days post-challenge. Therefore, in order to assess whether the CIV vaccine protects against CIV-induced lung consolidation, all dogs in control and vaccinated groups were euthanized on day 10 post-challenge, and necropsy was performed. Lung lesions were evaluated and scored as percent consolidation of each lung lobe. The percent consolidation of each lung lobe scored during necropsy was converted into weighted scores based on the lung scoring system for dogs (similar to Swine influenza virus lung scoring system in Diseases of the Swine (1999) 8th Ed., Ch. 61, p. 913-940). Median lung scores for vaccinated and control groups were compared using Wilcoxon Exact Rank Sum tests and two sided p-values were reported. Mitigated fraction estimate of vaccine efficiency relative to controls and the 95% confidence interval for the estimate was also reported.

All dogs in control group (100%) exhibited varying, degree of lung consolidation whereas only one dog in vaccinated group showed a mild lung consolidation (12.5%). Lung lesions in non-vaccinated control dogs were characterized by hemorrhages and reddish consolidation and hepatization. The lung scores in control group ranged between 0.10 and 14.70 with median lung score of 4.9. The lung scores in control group were significantly higher than that in vaccinated group (p=0.0005; mitigating fraction estimate is 93.5%). The lung scores unequivocally demonstrate that the CIV vaccine formulation used in this study protects dogs against CIV-induced lung consolidation.

In addition to scoring lesion during necropsy, lung tissues were also collected aseptically for virus isolation and in formalin for histopathology. There was no detectable CIV in lung tissue samples from both vaccinated ad control dogs, which correlated with the absence of nasal virus shedding. This was expected since influenza viruses cause acute infection with peak virus shedding and clinical signs within the first seven days. The virus was cleared completely from lung tissue by 10 days post-infection. These results are in agreement with the results of the previous study. Histopathological examination revealed varying degrees of histopathological changes suggestive of inflammation in lung tissues from control as well as vaccinated dogs. This finding was not unexpected because since immune response to any pathogen is likely to induce a certain degree of inflammatory response even in the presence of pathogen-specific immunity. Furthermore, the severity of histopathologic lung lesions in control and vaccinated dogs could not be compared since the tissues were not collected selectively from areas with lung lesions. Therefore, histopathology could not be used as a criterion to evaluate efficacy of the vaccine in this study.

### CONCLUSIONS

The vaccination induced a significantly higher antibody response, as determined by HAI assay, following first (GMT-22) and second vaccinations (GM T135) indicating stimulation of immune response by the vaccine.

The vaccine significantly decreased CIV-induced clinical signs, especially coughing, at a dose of 500 HAU per dog demonstrating effectiveness of the vaccine in controlling CIV-induced clinical disease.

The vaccine significantly reduced nasal virus shedding in vaccinated dogs, which demonstrates the efficacy of the vaccine in reducing infection.

The vaccine successfully protected dogs against CIV-induced lung consolidation proving the efficacy of the vaccine against the most severe clinical outcome, pneumonia.

The vaccine caused no major adverse reactions in dogs demonstrating the safety of the vaccine.

### Clinical Assessment Guide

### Nasal Discharge

### 0=Absent

0.5=Serous discharge: Water fluid dribbles from the nostril. Fluids running out of the nose are recorded here.
1=Mucopurulent discharge, mild to moderate: Cloudy fluid mixed with mucous runs at least halfway down from the nose to the mouth.
2=Mucopurulent discharge, severe: Mucous run past the mouth.

### Ocular Discharge

0=Absent: Small amount of dried crusted material in the corner of the eye is not considered an ocular discharge.
0.5=Serous discharge: Clear fluid discharge running outside of the eye.
1=Mucopurulent discharge, mild to moderate: Cloudy fluid mixed with mucous that runs at least halfway down from the eye to the mouth.
2=Mucopurulent discharge, severe. Fluid or mucous runs halfway down the nose or rim the eve and soak the hair at inner or outer corner of the eye.

### Cough

### 0=Absent

0.5=Mild: Only one brief cough is observed.
1.0=Moderate: Cough is persistent, occurring repeatedly in the observation period.
2.0=Severe. Cough is accompanied by choking or retching sounds.

### Sneezing

### 0=Absent

### 2=Present

### Dyspnea

0=Absent (Normal breathing)

### 2=Present (Panting)

### Depression

0=Absent (Normal activity)
2=Present: Dog is less active or playful, compared to normal. Dogs are recorded if lethargic or lying down and reluctant to stand while observation was conducted.

Although the foregoing invention has been described in detail for purposes of clarity of understanding, it will be obvious that certain modification may be practiced within the scope of the appended claims. All publications and patent documents cited herein are hereby incorporated by reference in their entirety for all purposes to the same extent as if each were so individually denoted.

From the foregoing it will be apparent that the invention provides for number of uses. For example, the invention provides for the use of any of newly identified pathogenic influenza virus strains for preparation of cell culture-adapted isolates and/or for vaccines as well as known pathogenic strains. The invention provides for the use of any newly identified cell culture cells that are or can be made permissible to influenza growth. The invention provides for the preparation of the tissue-culture adapted strains into vaccines having at least attenuated, subunit, split or killed virus, but also having adjuvants, carriers, excipients, anti-influenza pharmaceuticals, and other agents that increase the immune response to the virus. The vaccines can be used before contact with influenza or after contact.

What is claimed is:

1. A method of selecting a human influenza virus for growth on tissue culture cells by limiting dilution cloning, the method comprising:
serially diluting an influenza virus isolate and
contacting each dilution with cultured cells:
growing the cells for a time sufficient to produce cytopathic effects (CPE);
harvesting virus from the highest dilution that causes CPE; and
repeating the process with the harvested virus.

2. The method of claim 1 further comprising admixing the influenza virus isolate with an effective amount of trypsin before contacting the cultured cells.

3. The method of claim 2, wherein the trypsin is type IX trypsin.

4. The method of claim 2, wherein the step of contacting the tissue-culture adapted isolate with the tissue culture cells is carried out at an MOI of less than about 0.01.

5. The method of claim 1, wherein the tissue culture cells are mammalian embryonic kidney cells.

6. The method of claim 5, wherein the mammalian embryonic kidney cells are human embryonic kidney cells.

7. The method of claim 1, wherein the influenza virus is an influenza A, B, or C virus.

8. The method of claim 7, wherein the influenza A virus is an H5N1 strain.

9. The method of claim 1, wherein the influenza virus isolate is initially grown in embryonated eggs to obtain a large inoculum for adaptation to tissue culture.

10. The method of claim 9, wherein the influenza virus isolate is initially grown on the amniotic membrane.

11. A method for the production of a human influenza virus vaccine comprising purifying the harvested virus of claim 1.

12. The method of claim 11, wherein the step of purifying is carried out using size exclusion chromatography.

13. The method of claim 11, further comprising treating the virus with an amount of binary ethyleneimine (BEI) effective to inactivate the virus.

14. An immunogenic composition comprising a BEI-inactivated human influenza virus and an adjuvant.

15. A vaccine comprising a human influenza virus formulated at less than 4 μg of human influenza HA per dose wherein at least 70% of the HA has the same amino acid sequence.

16. The vaccine of claim 15, wherein the human influenza virus is inactivated by BEI.

17. The vaccine of claim 15, wherein the vaccine further comprises ISCOM.

18. The vaccine of claim 15, wherein at least 90% of the HA has the same amino acid sequence.

19. A method of selecting a canine influenza virus (CIV) H3N8 for growth on tissue culture cells by limiting dilution cloning, the method comprising:
serially diluting an influenza virus isolate and
contacting each dilution with cultured cells;
growing the cells for a time sufficient to produce cytopathic effects (CPE);
harvesting virus from the highest dilution that causes CPE; and
repeating the process with the harvested virus.

20. The method of claim 19, further comprising admixing the influenza virus isolate with an effective amount of trypsin before contacting the cultured cells.

21. the method of claim 19, wherein the trypsin is type IX trypsin.

22. The method of claim 19, wherein the tissue culture cells are mammalian embryonic kidney cells.

23. The method of claim 5, wherein the mammalian embryonic kidney cells are Madin-Darby bovine kidney (MDBK) cells.

24. An immunogenic composition comprising an inactivated canine influenza virus (CIV) H3N8 and an adjuvant.

25. The immunogenic composition of claim 24, wherein the adjuvant is an oil and water emulsion.

26. The immunogenic composition of claim 24, wherein the adjuvant is aluminum hydroxide.

27. The immunogenic composition of claim 24, wherein the inactivated CIV H3N8 is a binary ethyleneimine-inactivated CIV H3N8.

28. A vaccine comprising the immunogenic composition of claim 24.

29. The vaccine of claim 28 further comprising an immunologically effective amount of one or more additional inactivated CIV serotypes.

30. The vaccine of claim 28, further comprising an additional pathogen, wherein the additional pathogen is selected from the group consisting of canine distemper virus, canine adenovirus, canine parvovirus, canine parainfluenza virus, canine coronavirus, Leptospira serovars, Leishmania organisms, a Borrolia species (spp.); Bordetella bronchiseptica, Mycoplasma species, rabies virus, Ehrichia canis, and combinations thereof.

31. The vaccine of claim 28, wherein the CIV H3N8 is formulated at 500 HAU/dose and wherein at least 70% of the HA has the same amino acid sequence.

32. The vaccine of claim 31, wherein the adjuvant is aluminum hydroxide.

33. The vaccine of claim 34, wherein at least 90% of the HA has the same amino acid sequence.

34. A method of immunizing a canine against CIV comprising injecting the canine with the vaccine of claim 28.

35. Serum comprising antibodies that bind to CIV H3N8 obtained from a canine immunized by the method of claim 34
